(19) United States
(12) Patent Application Publication
(10) Pub. No.: US 2026/0133181 A1
(43) Pub. Date: May 14, 2026

(54) Universal Antibody Competition Equivalency Assay and Methods of Using

(71) Applicant: The Government of the United States, as Represented by the Director of the Defense Health Agency, Fort Detrick, MD (US)

(72) Inventor: Elke Bergmann-Leitner, Kensington, MD (US)

(21) Appl. No.: 19/370,411

(22) Filed: Oct. 27, 2025

Related U.S. Application Data
(60) Provisional application No. 63/719,862, filed on Nov. 13, 2024.

(51) Int. Cl. G01N 33/50; G01N 33/68

(52) U.S. Cl. CPC G01N 33/5091; G01N 33/6845; G01N 33/6854

(57) ABSTRACT

Disclosed herein is a universal species-independent method for characterizing antibodies present in a sample that may provide protective immunity against a given pathogen or antigen of interest.

### CROSS-REFERENCE TO RELATED APPLICATIONS

This application claims the benefit of U.S. Patent Application No. 63/719,862, filed Nov. 13, 2024, which is herein incorporated by reference in its entirety.

### ACKNOWLEDGEMENT OF GOVERNMENT SUPPORT

This invention was made by an employee of The Government of the United States, as represented by the Director of the Defense Health Agency. The Government has certain rights in the invention.

### REFERENCE TO A SEQUENCE LISTING SUBMITTED VIA PATENT CENTER

The content of the XML file of the sequence listing named “20251013_034047_082US1_ST26” which is 7,564 bytes in size was created on Oct. 13, 2025 and electronically submitted via Patent Center herewith the application is incorporated herein by reference in its entirety.

### BACKGROUND OF THE INVENTION

### 1. Field of the Invention

The field of the invention relates to immunity profiling and vaccine development.

### 2. Description of the Related Art

Vaccine design often targets pathogen-derived antigens previously identified as critical for inducing and mediating protective immune responses. The breadth or focus of serological responses to specific epitopes of the target antigens can determine whether the immune response is protective or not. Assays that can quantitatively evaluate epitope-specific immune responses are a powerful in vitro tool that can be employed to predict the efficacy of an experimental vaccine prior to controlled human infections or field efficacy studies.

### SUMMARY OF THE INVENTION

In some embodiments, the present invention is directed to a universal species-independent method (“ACE assay”) for characterizing unknown antibodies that may be present in a test sample, which comprises selecting a plurality of epitopes of a given antigen of interest, wherein one or more epitopes in the plurality of epitopes are known to induce a protective antibody response against the given antigen in a subject when the subject is vaccinated with the one or more epitopes; obtaining a reporter mixture comprising a plurality of benchmark antibodies, each capable of binding one or more epitopes in the plurality of epitopes when contacted therewith; obtaining an assay substrate comprising the plurality of epitopes immobilized on a first single assay well, wherein the epitopes immobilized on the first single assay well are capable of being distinguished from each other; mixing the test sample with an amount of the reporter mixture to give a test mixture; contacting the plurality of epitopes immobilized on the first single assay well with the test mixture and then detecting the formation of the one or more epitope-antibody conjugates; and characterizing the unknown antibodies in the test sample as being protective when the unknown antibodies equally compete with or outcompete the plurality of benchmark antibodies in the amount of the reporter mixture in forming epitope-antibody conjugates with the one or more epitopes known to induce a protective antibody response that are immobilized on the first single assay well. In some embodiments, the concentration of each benchmark antibody in the plurality of benchmark antibodies in the reporter mixture does not exceed its linear detection range, wherein the linear detection range of a given antibody is a concentration range in which equal competition between the given antibody and itself for binding its cognate ligand is detectable. In some embodiments, the linear detection range of each benchmark antibody is determined by conducting a plurality of competition assay experiments between benchmark antibody test mixtures and at least one benchmark antibody reporter mixture, wherein the benchmark antibody test mixtures have differing concentrations of the plurality of benchmark antibodies, and wherein the benchmark antibodies in the benchmark antibody reporter mixture are distinguishable from the benchmark antibodies in the benchmark antibody test mixtures. In some embodiments, the method comprises comparing the formed epitope-antibody conjugates with a reference. In some embodiments, the reference is the amounts of each benchmark antibody in the plurality of benchmark antibodies that equally compete with themselves for binding each epitope immobilized on a second single assay well, said second single assay well is a duplicate of the first single assay well. In some embodiments, the reference is a profile of epitope-antibody conjugates formed between antibodies of a reference-reporter mixture, said reference-reporter mixture comprising (1) a reference sample having the plurality of benchmark antibodies the same amount as in the reporter mixture that was mixed with the test sample, and (2) the same amount of the reporter mixture that was mixed with the test sample, wherein, when bound to one or more epitopes immobilized on second single assay well, said second single assay well is a duplicate of the first single assay well, the benchmark antibodies from the reference sample are distinguishable from the benchmark antibodies from the reporter mixture. In some embodiments, the method further comprises contacting the reference-reporter mixture with the plurality of epitopes immobilized on the second single assay well; and comparing the amounts of the benchmark antibodies from the reporter mixture that are bound to each epitope in the plurality of epitopes immobilized in the first single assay well to the amounts of either the benchmark antibodies of the reference sample or the benchmark antibodies in the reporter mixture that are bound to the plurality of epitopes immobilized on the second single assay well. In some embodiments, the method further comprises obtaining a standard curve for each epitope in the plurality of epitopes, wherein the standard curve of a given epitope represents the amounts of the plurality of the benchmark antibodies in the reporter mixture that outcompete themselves over several different concentrations and/or dilutions of the concentration of the plurality of the benchmark antibodies present in the reporter mixture and thereby bind the given epitope that is immobilized in single assay wells that are duplicates of the first single assay well. In some embodiments, the method further comprises obtaining a dose response curve of the test sample that represents different dilutions and/or concentrations of the test sample titrated against the reporter mixture. In some embodiments, the method further comprises obtaining a dose response curve of several different concentrations and/or dilutions of a reference sample titrated against the reporter mixture, wherein said reference sample comprises the same benchmark antibodies as in the reporter mixture and in the same concentrations. In some embodiments, each benchmark antibody in the plurality of benchmark antibodies in the reporter mixture have a detectable label.

The ACE assays disclosed herein may be used to obtain an antibody profile of a subject against a given pathogen or antigen of interest using a sample obtained from the subject as a test sample. The antibody profile may be obtained before and/or after administering to the subject a composition comprising the given antigen of interest or an antigenic epitope thereof. The antibody profile may be obtained before and/or after the subject is challenged or infected by a pathogen that expresses the given antigen of interest. The antibody profile may be obtained from a subject who has been administered the composition (e.g., immunized) before and/or after the subject is then challenged or infected with a pathogen that expresses the given antigen of interest or the antigenic epitope thereof.

In some embodiments, the present invention is directed to a kit comprising (a) (1) a plurality of antigenic epitopes of a given antigen, which each antigenic epitope is immobilized in an assigned location in each assay well of an assay substrate whereby each single assay well contains the plurality of antigenic epitopes immobilized at their own assigned location; or (2) the plurality of antigenic epitopes and linkers along with reagents for immobilizing each antigenic epitope in the plurality at an assigned location in each assay well of the assay substrate; (b) a reporter mixture comprising a plurality of benchmark antibodies; and (c) a reference. In some embodiments, the reference is a reference sample.

In some embodiments, the antigen of interest is the CSP molecule. In some embodiments, the plurality of epitopes comprise one or more of peptides having SEQ ID NO: 1, SEQ ID NO: 2, SEQ ID NO: 3, SEQ ID NO: 5, SEQ ID NO: 6, and/or SEQ ID NO: 7. In some embodiments the plurality of epitopes consist or consist essentially of SEQ ID NO: 1, SEQ ID NO: 2, SEQ ID NO: 3, SEQ ID NO: 5, SEQ ID NO: 6, and SEQ ID NO: 7.

In some embodiments, the present invention is directed to a universal species-independent method for characterizing unknown antibodies that may be present in a test sample, which comprises (a) selecting a plurality of epitopes of a circumsporozoite protein (CSP) of a Plasmodium spp, e.g., Plasmodium falciparum, wherein one or more epitopes in the plurality of epitopes are known to induce a protective antibody response against the given antigen in a subject when the subject is vaccinated with the one or more epitopes; (b) obtaining a reporter mixture comprising a plurality of benchmark antibodies, each capable of binding one or more epitopes in the plurality of epitopes when contacted therewith; (c) obtaining an assay substrate comprising the plurality of epitopes immobilized on a first single assay well, wherein the epitopes immobilized on the first single assay well are capable of being distinguished from each other; (d) mixing the test sample with an amount of the reporter mixture to give a test mixture; (e) contacting the plurality of epitopes immobilized on the first single assay well with the test mixture and then detecting the formation of the one or more epitope-antibody conjugates; and (f) characterizing the unknown antibodies in the test sample as being protective when the unknown antibodies equally compete with or outcompete the plurality of benchmark antibodies in the amount of the reporter mixture in forming epitope-antibody conjugates with the one or more epitopes known to induce a protective antibody response that are immobilized on the first single assay well. In some embodiments, the plurality of epitopes comprise one or more epitopes selected from SEQ ID NOs: 1 to 7. In some embodiments, the plurality of epitopes comprise or consist of SEQ ID NOs: 1 to 7. In some embodiments, the plurality of benchmark antibodies comprise one or more of the following: mAb 5D5, mAb CIS43, mAb L9, mAb 317, mAb 311, mAb 236, mAb 1512, mAb 369. In some embodiments, the plurality of benchmark antibodies comprise (a) mAb 5D5, (b) mAb CIS43, (c) mAb L9, (d) mAb 317 and/or mAb 311, and (e) at least one of mAb 236, mAb 1512, and mAb 369. In some embodiments, the plurality of benchmark antibodies consist of mAb 5D5, mAb CIS43, mAb L9, mAb 317, mAb 311, mAb 236, mAb 1512, and mAb 369. In some embodiments, the concentration of each benchmark antibody in the plurality of benchmark antibodies in the reporter mixture does not exceed its linear detection range, wherein the linear detection range of a given antibody is a concentration range in which equal competition between the given antibody and itself for binding its cognate ligand is detectable. In some embodiments, the linear detection range of each benchmark antibody is determined by conducting a plurality of competition assay experiments between benchmark antibody test mixtures and at least one benchmark antibody reporter mixture, wherein the benchmark antibody test mixtures have differing concentrations of the plurality of benchmark antibodies, and wherein the benchmark antibodies in the benchmark antibody reporter mixture are distinguishable from the benchmark antibodies in the benchmark antibody test mixtures. In some embodiments, step (f) comprises comparing the formed epitope-antibody conjugates with a reference. In some embodiments, the reference is the amounts of each benchmark antibody in the plurality of benchmark antibodies that equally compete with themselves for binding each epitope immobilized on a second single assay well, said second single assay well is a duplicate of the first single assay well. In some embodiments, the reference is a profile of epitope-antibody conjugates formed between antibodies of a reference-reporter mixture, said reference-reporter mixture comprising (1) a reference sample having the plurality of benchmark antibodies the same amount as in the reporter mixture that was mixed with the test sample, and (2) the same amount of the reporter mixture that was mixed with the test sample, wherein, when bound to one or more epitopes immobilized on second single assay well, said second single assay well is a duplicate of the first single assay well, the benchmark antibodies from the reference sample are distinguishable from the benchmark antibodies from the reporter mixture. In some embodiments, the method further comprises (i) contacting the reference-reporter mixture with the plurality of epitopes immobilized on the second single assay well; and (ii) comparing the amounts of the benchmark antibodies from the reporter mixture that are bound to each epitope in the plurality of epitopes immobilized in the first single assay well to the amounts of either the benchmark antibodies of the reference sample or the benchmark antibodies in the reporter mixture that are bound to the plurality of epitopes immobilized on the second single assay well. In some embodiments, the method further comprises obtaining a standard curve for each epitope in the plurality of epitopes, wherein the standard curve of a given epitope represents the amounts of the plurality of the benchmark antibodies in the reporter mixture that outcompete themselves over several different concentrations and/or dilutions of the concentration of the plurality of the benchmark antibodies present in the reporter mixture and thereby bind the given epitope that is immobilized in single assay wells that are duplicates of the first single assay well. In some embodiments, the method further comprises obtaining a dose response curve of the test sample that represents different dilutions and/or concentrations of the test sample titrated against the reporter mixture. In some embodiments, the method further comprises obtaining a dose response curve of several different concentrations and/or dilutions of a reference sample titrated against the reporter mixture, wherein said reference sample comprises the same benchmark antibodies as in the reporter mixture and in the same concentrations. In some embodiments, each benchmark antibody in the plurality of benchmark antibodies in the reporter mixture have a detectable label.

In some embodiments, the present invention is directed to a method of obtaining an antibody profile of a subject, which comprises obtaining a sample from the subject and using the sample as a test sample, then (a) selecting a plurality of epitopes of a circumsporozoite protein (CSP) of a Plasmodium spp, e.g., Plasmodium falciparum, wherein one or more epitopes in the plurality of epitopes are known to induce a protective antibody response against the given antigen in the subject when the subject is vaccinated with the one or more epitopes; (b) obtaining a reporter mixture comprising a plurality of benchmark antibodies, each capable of binding one or more epitopes in the plurality of epitopes when contacted therewith; (c) obtaining an assay substrate comprising the plurality of epitopes immobilized on a first single assay well, wherein the epitopes immobilized on the first single assay well are capable of being distinguished from each other; (d) mixing the test sample with an amount of the reporter mixture to give a test mixture; (e) contacting the plurality of epitopes immobilized on the first single assay well with the test mixture and then detecting the formation of the one or more epitope-antibody conjugates; and (f) characterizing the unknown antibodies in the test sample as being protective when the unknown antibodies equally compete with or outcompete the plurality of benchmark antibodies in the amount of the reporter mixture in forming epitope-antibody conjugates with the one or more epitopes known to induce a protective antibody response that are immobilized on the first single assay well. In some embodiments, the plurality of epitopes comprise one or more epitopes selected from SEQ ID NOs: 1 to 7. In some embodiments, the plurality of epitopes comprise or consist of SEQ ID NOs: 1 to 7. In some embodiments, the plurality of benchmark antibodies comprise one or more of the following: mAb 5D5, mAb CIS43, mAb L9, mAb 317, mAb 311, mAb 236, mAb 1512, mAb 369. In some embodiments, the plurality of benchmark antibodies comprise (a) mAb 5D5, (b) mAb CIS43, (c) mAb L9, (d) mAb 317 and/or mAb 311, and (e) at least one of mAb 236, mAb 1512, and mAb 369. In some embodiments, the plurality of benchmark antibodies consist of mAb 5D5, mAb CIS43, mAb L9, mAb 317, mAb 311, mAb 236, mAb 1512, and mAb 369. In some embodiments, the concentration of each benchmark antibody in the plurality of benchmark antibodies in the reporter mixture does not exceed its linear detection range, wherein the linear detection range of a given antibody is a concentration range in which equal competition between the given antibody and itself for binding its cognate ligand is detectable. In some embodiments, the linear detection range of each benchmark antibody is determined by conducting a plurality of competition assay experiments between benchmark antibody test mixtures and at least one benchmark antibody reporter mixture, wherein the benchmark antibody test mixtures have differing concentrations of the plurality of benchmark antibodies, and wherein the benchmark antibodies in the benchmark antibody reporter mixture are distinguishable from the benchmark antibodies in the benchmark antibody test mixtures. In some embodiments, step (f) comprises comparing the formed epitope-antibody conjugates with a reference. In some embodiments, the reference is the amounts of each benchmark antibody in the plurality of benchmark antibodies that equally compete with themselves for binding each epitope immobilized on a second single assay well, said second single assay well is a duplicate of the first single assay well. In some embodiments, the reference is a profile of epitope-antibody conjugates formed between antibodies of a reference-reporter mixture, said reference-reporter mixture comprising (1) a reference sample having the plurality of benchmark antibodies the same amount as in the reporter mixture that was mixed with the test sample, and (2) the same amount of the reporter mixture that was mixed with the test sample, wherein, when bound to one or more epitopes immobilized on second single assay well, said second single assay well is a duplicate of the first single assay well, the benchmark antibodies from the reference sample are distinguishable from the benchmark antibodies from the reporter mixture. In some embodiments, the method further comprises (i) contacting the reference-reporter mixture with the plurality of epitopes immobilized on the second single assay well; and (ii) comparing the amounts of the benchmark antibodies from the reporter mixture that are bound to each epitope in the plurality of epitopes immobilized in the first single assay well to the amounts of either the benchmark antibodies of the reference sample or the benchmark antibodies in the reporter mixture that are bound to the plurality of epitopes immobilized on the second single assay well. In some embodiments, the method further comprises obtaining a standard curve for each epitope in the plurality of epitopes, wherein the standard curve of a given epitope represents the amounts of the plurality of the benchmark antibodies in the reporter mixture that outcompete themselves over several different concentrations and/or dilutions of the concentration of the plurality of the benchmark antibodies present in the reporter mixture and thereby bind the given epitope that is immobilized in single assay wells that are duplicates of the first single assay well. In some embodiments, the method further comprises obtaining a dose response curve of the test sample that represents different dilutions and/or concentrations of the test sample titrated against the reporter mixture. In some embodiments, the method further comprises obtaining a dose response curve of several different concentrations and/or dilutions of a reference sample titrated against the reporter mixture, wherein said reference sample comprises the same benchmark antibodies as in the reporter mixture and in the same concentrations. In some embodiments, each benchmark antibody in the plurality of benchmark antibodies in the reporter mixture have a detectable label. In some embodiments, the method further comprises administering to the subject a composition comprising the given antigen of interest or an antigenic epitope thereof before and/or after obtaining the sample from the subject. In some embodiments, the method further comprises challenging the subject with a pathogen that expresses the CSP before and/or after obtaining the sample from the subject. In some embodiments, the method further comprises challenging the subject with a pathogen that expresses the given antigen of interest before and/or the subject is administered the composition.

In some embodiments, the present invention is directed to a kit comprising (a) (1) a plurality of epitopes of the circumsporozoite protein (CSP) of a Plasmodium spp, e.g., Plasmodium falciparum, which each antigenic epitope is immobilized in an assigned location in each assay well of an assay substrate whereby each single assay well contains the plurality of antigenic epitopes immobilized at their own assigned location; or (2) the plurality of antigenic epitopes and linkers along with reagents for immobilizing each antigenic epitope in the plurality at an assigned location in each assay well of the assay substrate; (b) a reporter mixture comprising a plurality of benchmark antibodies; and (c) a reference. In some embodiments, the plurality of epitopes comprise one or more epitopes selected from SEQ ID NOs: 1 to 7. In some embodiments, the plurality of epitopes comprise or consist of SEQ ID NOs: 1 to 7. In some embodiments, the plurality of benchmark antibodies comprise one or more of the following: mAb 5D5, mAb CIS43, mAb L9, mAb 317, mAb 311, mAb 236, mAb 1512, mAb 369. In some embodiments, the plurality of benchmark antibodies comprise (a) mAb 5D5, (b) mAb CIS43, (c) mAb L9, (d) mAb 317 and/or mAb 311, and (e) at least one of mAb 236, mAb 1512, and mAb 369. In some embodiments, the plurality of benchmark antibodies consist of mAb 5D5, mAb CIS43, mAb L9, mAb 317, mAb 311, mAb 236, mAb 1512, and mAb 369.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

The patent or application file contains at least one drawing executed in color. Copies of this patent or patent application publication with color drawing(s) will be provided by the Office upon request and payment of the necessary fee. This invention is further understood by reference to the drawings wherein:

FIG. 1: Exemplary overview of ACE assays. A 10-spot assay well accommodates up to ten different antigens per well. Each epitope of a plurality of epitopes was immobilized in a single assay well at individually assigned spots (in the detailed experiments exemplified herein, spots 4-6 were not used). A serially diluted test sample was mixed with a reporter mixture, e.g., the Sulfo-Tag Reporter Mix, and then the test-reporter mixture was added to the single assay well. Loss/reduction in signal (e.g., luminescence) is seen when antibodies of the test sample successfully competed with benchmark antibodies in the reporter mixture.

FIG. 2: Schematically shows an ACE assay workflow.

FIG. 3: Serological profile of RTS,S vaccinees (NCT00075049) as determined using the ACE assay. Data expressed as ng/ml epitope-specific antibody concentration in sera of protected (n=18, (panel A)) or non-protected (n=18, (panel B)) RTS,S vaccinees using mAbs CIS43, 317, 236, and 369. Results for the N-terminus, which is not included in the RTS,S vaccine, are omitted since no reactivity of the sera was measured. Asterisks indicate statistical significance between protected vs. non-protected RTS,S vaccinees (two-sample T-test, *p<0.05, **<0.005).

FIG. 4: Principal component analysis (PCA) based on the integration of all ACE assay data identifies driving parameters of a protective response. Data from FIG. 3, panel A, were used for the PCA. The ellipses visualize the protective (blue) and non-protective (red) serological profile established using the ACE data. The diverging directions and length of the loading vectors visualize the contribution of the respective parameters to the non-protective vs. protective profiles and thereby exemplifies the importance and value of ACE assay: the ACE assay data pertaining to the central repeat region (mAb 317) and the C-terminus (i.e., C.term (3D7), H18, and H50) pointing in the horizontal direction and account for 70.34% (PC1) of the protective profile. mAb L9 has been tested in field studies and shown to be protective. The ACE assay data reveal that antibodies induced by the RTS,S vaccine to the L9 epitope, however, do not significantly contribute to protection (loading vector points in the vertical direction). No significant differences in the magnitude of these antibodies between protected and non-protected individuals were measured. The arrows from top to bottom are responses to the 317, H18, H50, 3D7 (C-term), CIS43, and L9 epitope.

FIG. 5: Antibody profile to CSP regions of protected vs. non-protected RTS,S immunized vaccinees. Correlation matrices indicate the relationship between the equivalencies of the antibody responses to the various CSP epitope specific mAbs. The color and sizes of the dots (scale next to graphs) indicate the degree of correlation between the different CSP peptides (small to large indicating low to high correlation). Correlation matrices stratified by protective status (left=protected, right=non-protected individuals). Red framed boxes highlight significant correlations.

FIG. 6: Agreement between ELISA- and ACE (Equivalence) results dependent on protective status. Log-transformed ELISA titers (y-axis) and Equivalence (x-axis) results for repeat-specific and C-terminal responses were stratified by protective status: responses of sera from protected (n=18, top panels) and non-protected individuals (n=18, bottom panels).

FIG. 7: Defining the specificity and competition concentration for the Sulfo-Tag Reporter Mix and Reference Sample. Panel A: Monoclonal antibodies were tested for reactivity with the seven plate antigens. Intensity of shading represents reactivity of the antigen. mAb 311 was not considered for further development due to lack of specificity. Below panel A: schematic of the CSP protein visualizing the regions represented by the plate antigens (peptides) used in this study. Panel B: Matrix experiments were performed titrating the concentration of each benchmark antibody in both the Reference Sample and the Sulfo-Tag Reporter Mix, to determine the concentration of each antibody at which 50% inhibition of the signal with the antibody's Sulfo-tagged “self” is achieved. Arrow indicates the condition which was selected for the assay.

FIG. 8: Structure of the C-terminal α-TSR domain of CSP (PDB: 3VDJ) is shown in colors (grey for linker to repeat region, purple=α-TSR region III, turquoise=CSP-flap) with polymorphic sites highlighted in red. The diagram emphasizes the importance of dissecting the antibody responses to the different regions of the CSP C-terminus using the ACE assay: (a) Functionally-active antibodies (such as mAb 369) bind to the conserved region of the TSR; (b) antibodies binding to the conserved portion of the C-terminus are associated with protective immunity.

FIG. 9: Serological profile of preclinical and clinical samples immunized with FMP014. Equivalency of serological responses compared to the standard testing panel (mAbs 5D5, CIS43, L9, 317, 236/369) to determine the differences in the antibody profiles in preclinical (Panel A, n=8 mice), non-human primate (Panel B, n=7 NHP) and clinical samples (Panel C, n=9 clinical samples). Boxplots represent the equivalency to epitope-specific mAbs (expressed as ng/mL). The data demonstrate the ability of using an ACE assay to assay test samples from different species (here: mouse, non-human primate, human) using the same assay reagents (i.e., the same immobilized epitopes, benchmark antibodies, reporter mixture, and reference). That is, no species-specific reagents are needed for assaying a plurality of subjects of different species to characterize their immunity against a given pathogen or antigen of interest. As such, ACE assays may be used to assay test samples from a plurality of subjects of different species in the same experiment, e.g., on the same assay substrate (e.g., 96-well assay substrate) using the same benchmark antibodies, reporter mixture, and reference for each test sample.

FIG. 10: An exemplary graph showing the % inhibition per μg/mL of mAb 317. A CSP-immune human serum pool was used as test sample (1:1000 dilution) to determine the inhibition of mAb 317 to bind to repeat specific plate antigens (minor-major and NANP). Here, the data demonstrate that mixing the test sample with the competing mAb (T+R) and then adding to the assay well provides a better dose response compared to adding the test sample first, incubating for one hour and then adding the competing mAb (T->R). Therefore, ACE assays preferably comprise mixing the test sample with the competing mAbs (benchmark antibodies) and then adding the test-reporter mixture to a single assay well.

FIG. 11: An exemplary graph showing that mAb 1512 does not sufficiently compete with a test sample. As in FIG. 10, a CSP-immune human serum pool was used as the test sample (1:1000 dilution) to determine the inhibition of mAb 1512's ability to bind to C-terminus specific plate antigens (3D7, H18, H50). Competition was performed by either adding the test sample first (T->R) and then the competing mAb to the assay plate or mixing the test sample with the competing mAb and then adding to the plate (T+R). It is noted that the likely reason mAb 1512 is not able to compete is because antibodies (either benchmark antibodies or antibodies in the test sample) binding the front side of the C-terminus lead to structural changes that prevent mAb 1512 from recognizing and binding to its cognate epitope in the backside of the C-terminus. Thus, these results indicate that ACE assays may be used to screen for immune interference, e.g., screen antibody cocktail mixtures for antibodies that will enhance or reduce the intended therapeutic outcome, i.e., the degree of antibody binding to a given target pathogen or antigen.

FIG. 12: Robustness of ACE assay performance. A dose response curve (“standard curve”) was established by titrating the Reference Sample in the same dilution scheme as the test samples for each epitope employed in the ACE assay. Such standard curves represent different dilutions and/or concentrations of a reference sample titrated against the given concentration of the reporter mixture that is employed in the ACE assay and may be used to monitor and/or standardize an ACE assay when the assays of test samples are not performed concurrently. The standard curves of FIG. 12, one for each epitope, were used to monitor the daily performance of the ACE assays as described in the detailed experiments herein to ensure reproducibility and repeatability of the assay. Error bars represent variability over 20 independent experiments.

FIG. 13: Representative dose response curve for CSP-immune test sample when competing with mAb CIS43 (top) and its calculated IC50 (bottom). A test sample (human serum pool was tested at 5 dilutions for its ability to inhibit binding of mAb CIS43 to its epitope (Junction). To enable an easy comparison between test samples and the antibodies therein to block the binding of epitope-specific benchmark antibodies, the IC50 (i.e., the test sample dilution that results in 50% inhibition of binding by benchmark antibodies) was chosen as the benchmark. The 50% signal reduction based on the titration of the sample (point on Y-axis) was determined and then the serum dilution (point on the x-axis) that corresponds to the 50% signal reduction was extrapolated using methods in the art. Such a dose response curve of a test sample represent different concentrations and/or dilutions of the test sample titrated against the given concentration of the reporter mixture that is employed in the ACE assay and represents the “efficacy” of the test sample against the given pathogen or antigen of interest. A dose response curve of a test sample allows one to determine the IC50 of the test sample. Thus, a dose response curve of a test sample, e.g., sera sample, may be used to determine the amount of the test sample that should be administered to confer sufficient passive immunity against the given pathogen or antigen of interest.

FIG. 14: Converting IC50 to equivalence. Using the standard curve established with the Reference Sample (FIG. 12), the IC50 of each test sample was converted into the concentration of epitope-specific antibodies (equivalence). To this end, the dose response curve of each benchmark antibody in the Reference Sample was plotted. A 4-point linear regression curve and its fit parameters (top) and statistics (bottom) are shown. Using this curve, the IC50 for each test sample (determined as shown in FIG. 13) was then backfitted to convert the IC50 from its serum dilution into equivalence (ng/mL). Such a linear regression curve represents different dilutions and/or concentrations of a reference sample titrated against the given concentration of the reporter mixture employed in the given ACE assay. Such a curve may be used to convert the IC50 of a given dilution (or concentration) determined for a given test sample into the ng/ml of antibodies in the test sample that are equivalent to the amount of benchmark antibodies the reporter mixture.

FIG. 15: Serological profile of preclinical and clinical vaccine recipient samples established by performing the ACE assay. The multiplex nature of ACE assays simultaneously measures the equivalence to each epitope-specific benchmark antibody in the test sample and enables direct comparisons between different subjects (including those of different species), different vaccines, different vaccine doses, different booster regimens, etc. Clinical samples: three individual RTS,S immune serum samples (RTS,S #1, #2, #3), three individual FMP13 immune serum samples (FMP13 #1, #2, #3), human serum pool from individuals living in malaria-endemic area (Kenyan Pool). Preclinical samples: RTS,S rabbit serum pools (high dose, low dose), RTS,S rat serum pool (high dose, low dose), and RTS,S mouse serum pool (one vaccine dose).

FIG. 16: Serological profile of test samples from a malaria field study (“BakMal”). Pooled serum samples (pooling was done based on low/medium/high ELISA titers to either CSP (“FL”), CSP C-term (“C-term”), or central repeat region (“NANP”). Data demonstrate that the ACE has sufficient sensitivity to measure equivalence in samples from donors that have not received a CSP-vaccine but have only seen the pathogen which typically results in low-magnitude serological responses. That is ACE assays exhibit superior sensitivity which may be used to distinguish between subjects who have been actively vaccinated against a given pathogen or antigen of interest and subjects who were not vaccinated but previously exposed (e.g., naturally infected by) to the given pathogen or antigen.

FIG. 17: Evolution of serological profiles in the course of vaccination. Serum samples from protected (n=17) and non-protected (n=14) RTS,S vaccinees immunized three times (p1=post 1st vaccination (top), p2=post 2nd vaccination (middle), p3=post 3rd vaccination/day of challenge (bottom)). Serum samples were diluted 1:1000 and assayed via the ACE assay to establish serological profiles. The ACE assay identifies and defines (A) epitope specificities of the CSP regions for the three time points for both the protected and non-protected groups; (B) significant difference between the protected and non-protected subjects for p3 (pre-challenge) for the C-terminus region; (C) the evolution of a protective immune response by refocusing the antibody response from the repeat region (mAb 317 equivalence) towards a C-terminal response after the second booster immunization and a further maturation of the C-terminal response after third vaccination towards the H18 and H50-epitopes. These results show that ACE assays may be used to monitor and/or optimize vaccine regimens, e.g., booster schedules. These results also show that ACE assay may be used to elucidate the evolution and immunodominance of the various regions of a given pathogen or antigen of interest by collecting and assaying test samples at several different points in time.

FIG. 18: Radar plots summarize longitudinal changes in the multi-dimensional antibody profiles of the protected and not-protected subjects in FIG. 17. Data expressed as natural log (In) transformed ng/mL. Spikes of radar plot represent plate antigens, colored lines show time points after each vaccination (post 1st=p1, post 2nd=p2, and pre-challenge/post 3rd=p3).

FIG. 19 to FIG. 21: Profiling antibody responses induced by different doses of a full length CSP-based mRNA construct and comparison of protective immunity. FIG. 19: Challenge model performed on immunized mice demonstrates the lower parasite burden especially in mice immunized with 1 μg/ml CSP mRNA. FIG. 20: ACE assays show the differences in the quality and quantity of the antibody responses induced by the CSP mRNA compared to that induced by the RTS,S/AS01 vaccine. FIG. 21: Reduction in liver burden. Percent inhibition of in vitro infection of cultured hepatocytes as measured by qPCR.

FIG. 22: ACE assays performed on different subunit mRNA constructs using the benchmark antibodies provided in Table 1. Left panel: epitope mapping results for the various constructs. Right panel: epitope mapping of reference immunizations (RTS,S at two doses) using the indicated mRNA construct (FL-PfCSP/LNP1) for comparison. ACE assays were performed as described herein.

FIG. 23 to FIG. 26: Longitudinal changes in human antibody profiles induced by the FMP13/ALFQ vaccine candidate. C-term=3D7. FIG. 23: Serological equivalence/antibody profile induced by standard regimen (i.e., three immunizations at 0, 1, and 2 months) of FMP13/ALFQ. Due to the low sample size of protected individuals (n=2/9) the box-plots for the protected profile are only suggesting that a C-terminal response seems important. FIG. 24: Serological equivalence/antibody profile induced by delayed fractional dose regimen (i.e., three immunizations at 0, 1, and 7 months) of FMP13/ALFQ. FIG. 25: Serological equivalence/antibody profile induced by delayed full dose regimen of FMP13/ALFQ. Highest vaccine efficacy with 1/9 not protected, 3/9 significant delay in parasitemia, and 5/9 sterilely protected. The antibody profile comparing protected vs. non-protected subjects demonstrated that protected subjects have a unique profile compared to the other cohorts. Interestingly, antibodies to the minor repeats and the cross-reactive C-terminal antibodies to H50) were significantly higher in protected individuals. The efficacy of the delayed fractional dose was lower compared to the delayed full dose regimen, i.e., 1 fully protected subject, 2 subjects with significant delay in parasitemia, and 4 non-protected subjects. Due to the high number of subjects with delayed vs sterile protection, the antibody responses were stratified further (FIG. 26). FIG. 26: Serological equivalence/antibody profiles differ between subjects fully protected vs subjects with significant delay in parasitemia. Time point “post 2” demonstrated greatest differences between the three groups. ACE assays were performed as described herein.

FIG. 27 to FIG. 29: Comparison of ACE assay antibody profiles induced in the indicated subjects by RTS,S/AS01B (FIG. 27), FMP13 (recombinant full length CSP protein adjuvanted with ALFQ, FIG. 28), and FMP14/ALFQ (self-assembling nanoparticle that displays repeat and C-terminus of CSP, FIG. 29). Pf16=3D7. Preclinical and clinical samples collected after the 2 weeks after the last immunization were tested using ACE assays. Immunization details: (1) RTS,S: 5 μg/dose for mice, rabbits, and rats; 50 μg/dose for humans, (2) FMP13: 5 μg/dose in mice; 20 μg/dose in nonhuman primates (NHP) and humans, and (3) FMP14: 5 μg/dose in mice; 20 μg/dose in nonhuman primates (NHP) and humans. All vaccinees received three immunizations at 1-month intervals. ACE assays were performed as described herein.

### DETAILED DESCRIPTION OF THE INVENTION

Disclosed herein is a multiplex competition assay method that may be used to map and measure the specificity and affinity of a subject's antibodies to one or more antigenic epitopes of a given pathogen or antigen and thereby characterize the degree of immunity the subject has against the given pathogen or antigen as compared to the specificities and affinities of given benchmark antibodies. As used herein, a “benchmark antibody” is an antibody in which its specificity and binding affinity against a given target pathogen or antigenic epitope thereof is known and is used as the benchmark measure of competitive binding to the target. That is, assay method as described herein characterizes antibody immunity by antibody competition equivalency and is therefore referred to herein as an “ACE assay”. ACE assays may be used to characterize the immunity of any subject against any given pathogen or antigen so long as at least one benchmark antibody against the given pathogen or antigen exists and can be compared with any antibodies in a given test sample, e.g., a serum sample, from the subject via a competition binding immunoassay. Test samples may be of any type, e.g., serum, plasma, saliva, breastmilk, feces, urine, tears, and other bodily fluids, laboratory created samples, etc. Test samples need only be suspected of containing antibodies which one desires to assess their equivalence to one or more given benchmark antibodies of interest.

The basic steps of ACE assays are (1) selecting a plurality of epitopes of interest of a given pathogen or antigen of interest; (2) immobilizing each epitope of interest to its own identifiable location in a single assay well of an assay substrate surface; (3) adding a given test sample, e.g., a sera sample of interest, and a mixture of benchmark antibodies to the single assay well, wherein the mixture comprises at least one benchmark antibody per each epitope in the plurality that the at least one benchmark antibody specifically binds with a known affinity; (4) detecting and quantifying the amount of each benchmark antibody bound to each epitope of interest, and comparing to that of a given reference.

In some embodiments, each benchmark antibody comprises its own detectable label that distinguishes it from other benchmark antibodies in the mixture. In some embodiments, each epitope of interest comprises a detectable label that is quenched when conjugated to the at least one benchmark antibody that specifically binds it via a quenching molecule bound to the at least one benchmark antibody.

In some embodiments, the given reference is a “reference sample”, which is a composition that comprises a mixture of the same antibodies and in the amount as the benchmark antibodies that are added to a test sample. As used herein, “reference antibodies” refer to the antibodies of a reference sample. In some embodiments, the reference antibodies lack a detectable label (where the benchmark antibodies have a detectable label) or lack a quenching molecule (where the benchmark antibodies have a quenching molecule). In some embodiments, the reference sample is a plurality of different dilutions. In some embodiments, the plurality of different dilutions of the reference sample comprise at least 8 different dilutions. In some embodiments, the plurality of different dilutions of the reference sample are used to create a standard curve. In some embodiments, the standard curve is used to convert a measured antibody-epitope IC50 into a concentration of the epitope-specific antibody present in the given test sample. In some embodiments, the reference is a profile of the amounts or concentrations of each benchmark antibody that should remain bound to each epitope of interest, which amounts or concentrations have been determined to be indicative of protective immunity against the given pathogen or antigen of interest. In some embodiments, the reference is a profile of the amounts or concentrations of each benchmark antibody that should be outcompeted, i.e., unbound, to its epitope of interest as a result of antibodies in the given test sample being bound thereto, which amounts or concentrations have been determined to provide protective immunity against the given pathogen or antigen of interest.

As used herein, a “reporter mixture” generically refers to a composition comprising the one or more benchmark antibodies, which when bound to a target epitope(s) immobilized in a single assay well the one or more benchmark antibodies of the reporter mixture are distinguishable from any antibodies in the test sample and any reference antibodies when they are bound to target epitope(s) immobilized in the single assay well. A “reference-reporter mixture” refers to a mixture of the reference sample and reporter mixture, preferably in a 1:1 volume ratio. A “test-reporter mixture” refers to a mixture of the test sample and the reporter mixture.

In some embodiments, ACE assays further include, using methods in the art (e.g., hybridoma and/or recombinant techniques) to (a) obtain one or more of the at least one benchmark antibody, and/or (b) determine its binding specificity and/or binding affinity, against a given antigenic epitope of interest. Methods such as those described in Beutler, et al. (2022) and Wang, et al. (2021) may be used to obtain and characterize antibodies that are suitable for use as a benchmark antibody against, e.g., a target epitope of a Plasmodium sp., such as Plasmodium falciparum, or any other pathogen or antigen of interest. Preferably, one or more benchmark antibodies used in a given ACE assay is a ‘monoclonal’ antibody (mAb) in the sense that the one or more benchmark antibodies have at least the same CDR amino acid sequences and therefore recognize and bind the same target epitope with substantially the same affinities and avidities. Nevertheless, the benchmark antibodies for the same given target epitope need not have the same exact CDR sequences or the exact same VH and VL sequences, so long as they bind the same given target epitope with the same specificity and avidity. That is, the amino acid sequences, including the sequences of the CDR regions, of the antibodies used as a benchmark antibody against a given target epitope need not be known so long as their binding specificities and binding affinities against the given target epitope are known and statistically determined (using methods in the art) as having the same binding specificities and affinities as if the antibodies shared the same sequences. Thus, a given benchmark antibody against a given target epitope may be polyclonal antibodies that have been affinity purified, using methods in the art, such that the polyclonal antibodies recognize and bind the given target epitope with essentially the same specificity and affinity.

In some embodiments, the ACE assays further include assaying the given test sample in at least four different dilutions. In some embodiments, the at least four different dilutions are three-fold dilutions. In some embodiments, the three-fold dilutions are used to generate a dose response curve.

In some embodiments, the ACE assays further include determining a linear detection range for each benchmark antibody. In some embodiments, the linear detection range is generated by calculating a dose response curve for competition between the unlabeled and labeled forms of the given benchmark antibody whereby the % recovery=(Calculated Concentration/Actual Concentration)*100) is between 80-120%.

In some embodiments, the ACE assays further include backfitting the measured antibody-epitope IC50 of the given test sample to a standard curve of IC50's of a plurality of different dilutions of the reference sample to obtain a concentration of the epitope-specific antibodies present in the given test sample. In some embodiments, the concentration is multiplied by the given dilution of the given test sample to obtain an effective antibody concentration which is the concentration of the antibody in the given test sample is equivalent to the given benchmark antibody in binding the given target epitope.

The equivalencies of antibodies in the test samples are calculated herein as an IC50 to provide comparative measurements between the test samples. The IC50 is the test sample dilution needed to equally compete with the benchmark antibodies for binding to the given epitopes. Specifically, an IC50 indicates the concentration of antibodies in a given test sample needed to outcompete 50% of a given benchmark antibody for binding the given target epitope. The IC50s may be converted into an absolute concentrations of epitope-specific antibodies within the given test sample.

In some embodiments, ACE assays may be used to characterize a subject's immunity against a given pathogen of interest. In these embodiments, when an ACE assay indicates the antibodies in a sera sample obtained from the subject are at least equivalent, e.g., have about the same or better specificity and binding affinity as that of benchmark antibodies against the same antigenic epitopes that are known to provide protection against the given pathogen, the subject's immunity is characterized as being protective. Conversely, when an ACE assay indicates the antibodies in a sera sample obtained from the subject fail to have about the same or better specificity and binding affinity as that of benchmark antibodies against the same antigenic epitopes that are known to provide protection against the given pathogen, the subject's immunity is characterized as being unprotected.

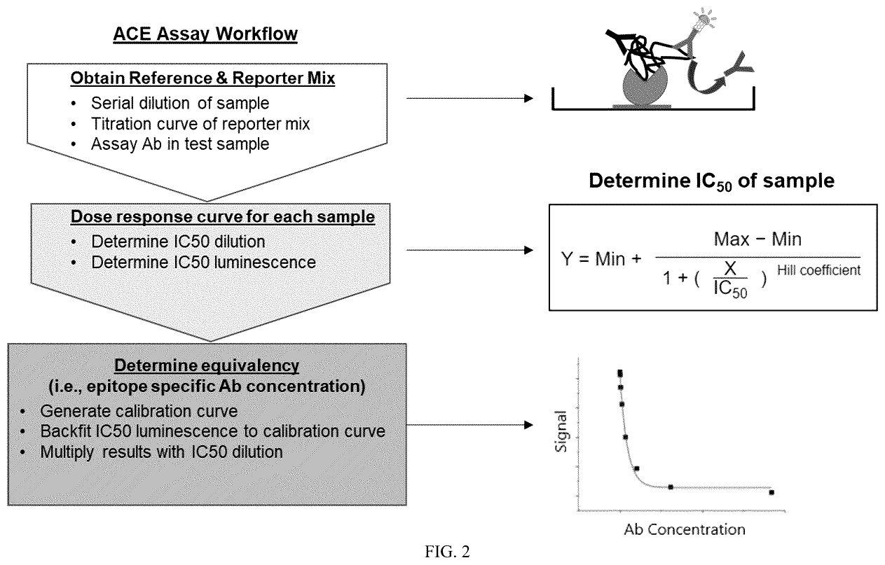

FIG. 2 schematically shows an ACE assay. First, plurality of given epitopes of a given pathogen or antigen of interest are selected along with a plurality benchmark antibodies, which each are capable of binding one or more epitopes in the plurality of epitopes when contacted therewith. Test samples and a reference sample are serially diluted, mixed with a reporter mixture (containing the plurality of benchmark antibodies, which are distinguishable from the benchmark antibodies in the reference sample) and added to an assay well having the plurality of epitopes immobilized thereon in a manner that allows their individual identification, e.g., immobilized at assigned locations in the assay well. While the test samples, reference sample, and reporter mixture may be serially added to the assay well, mixing them together before adding to the assay well resulted in comparatively superior sensitivity (FIG. 10). Like the test sample, the reference sample is serially diluted to generate a standard curve that may be used to monitor assay performance and inter-assay comparability (see, e.g., the dose response curve of FIG. 12). Upon detecting and quantifying the resulting epitope-antibody conjugates in the assay well, the equivalence of antibodies in the test sample is determined.

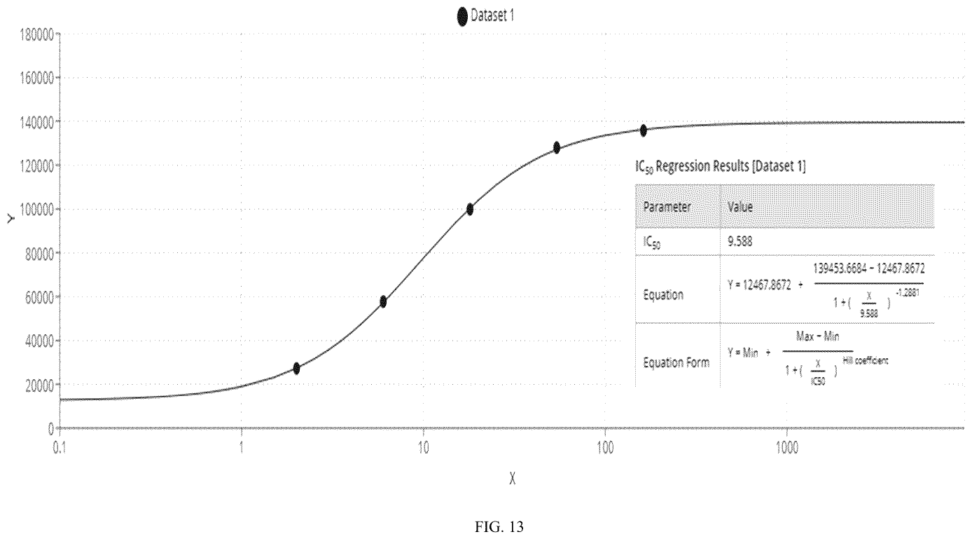

Briefly, a dose response curve is plotted for each test sample (see, e.g., FIG. 13, plotting test sample dilution vs. signal strength) and the sample dilution is determined that corresponds to inhibiting the binding of 50% of the benchmark antibodies (i.e., IC50) for each of the epitopes in the plurality of epitopes. The IC50 values (test sample dilutions) may be used directly as the measure of antibody equivalency or the IC50 values may be converted into a concentration (e.g., ng/ml) of epitope-specific antibody present in the test sample.

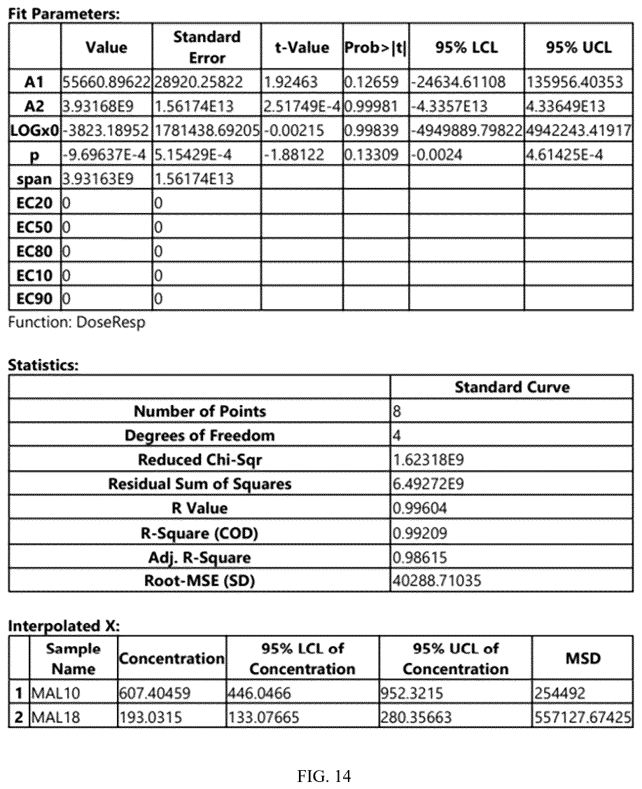

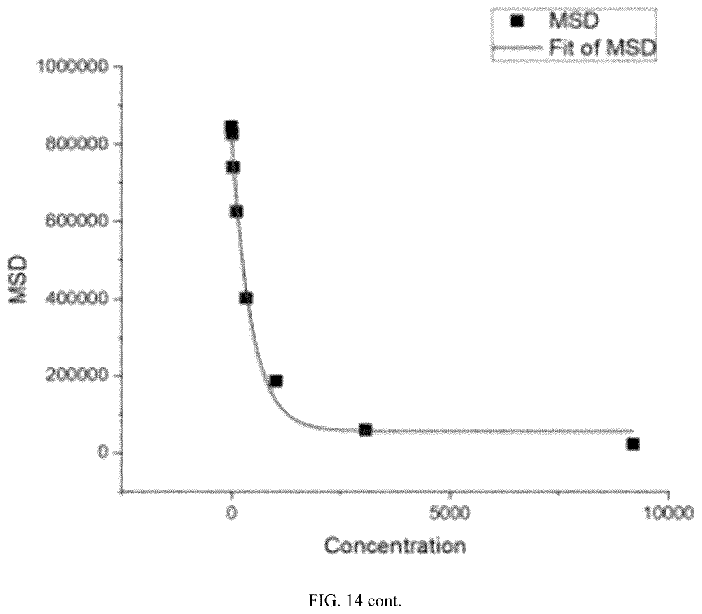

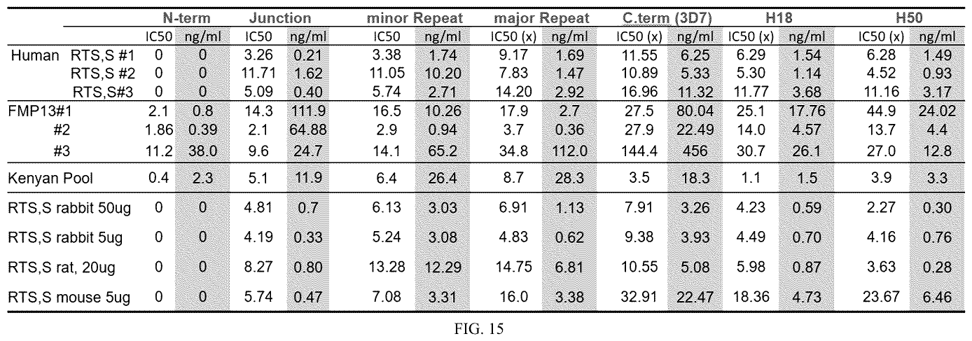

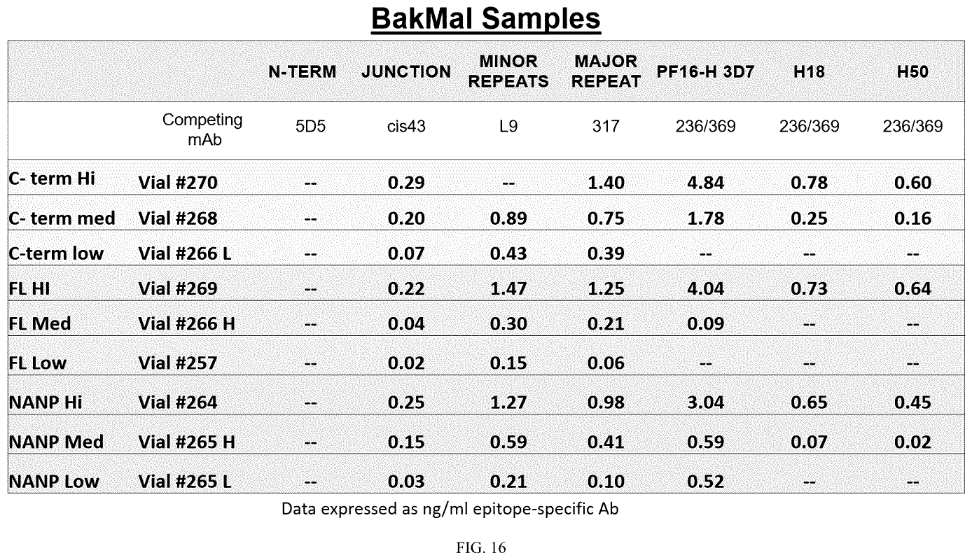

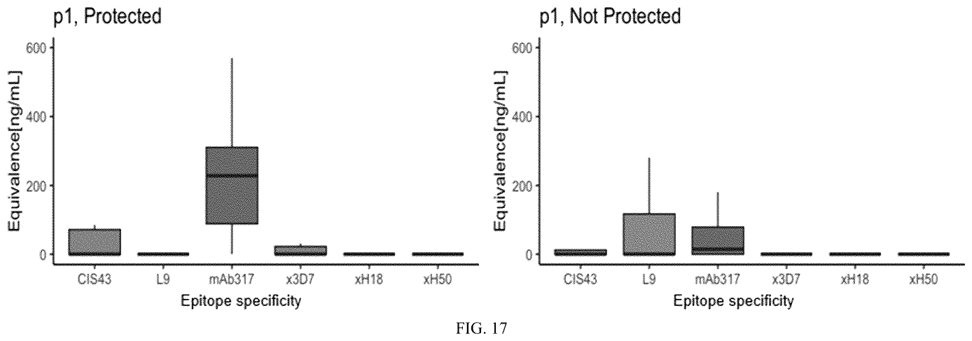

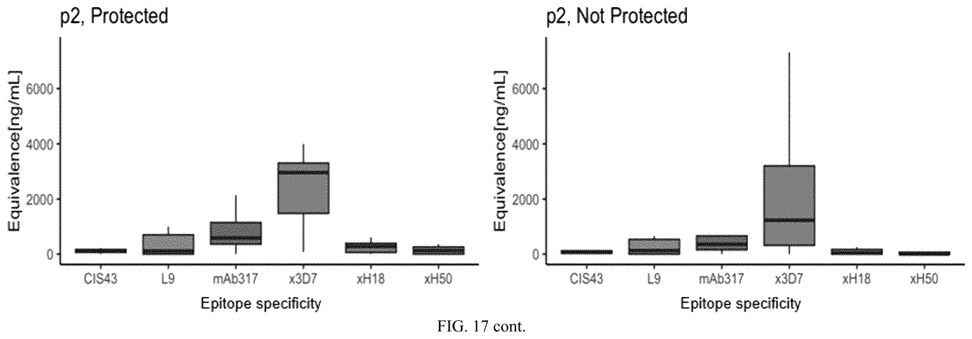

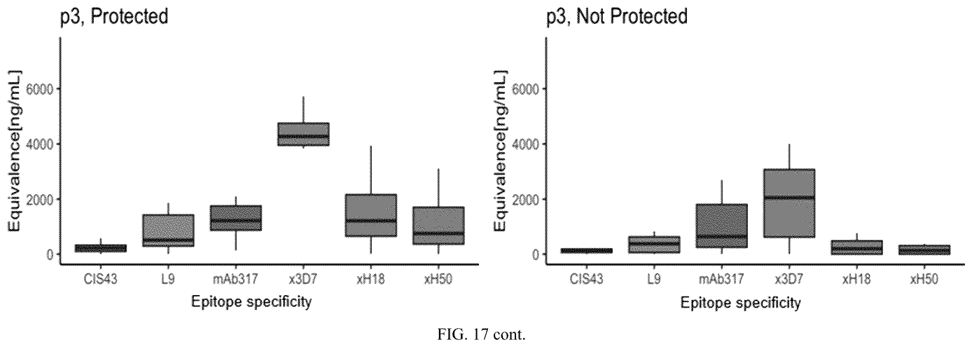

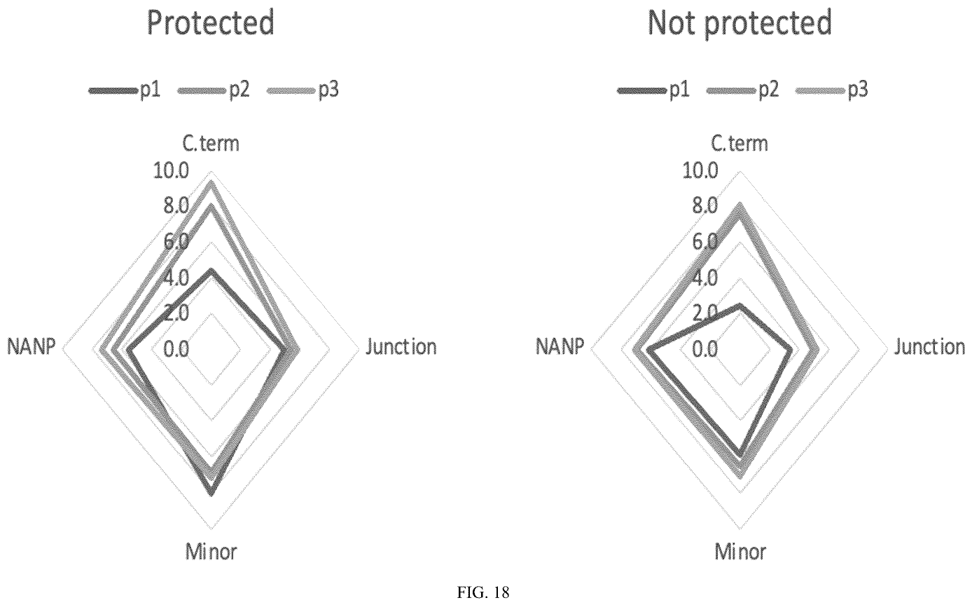

To convert an IC50 value into a concentration, the IC50 is backfitted onto a standard curve (see, e.g., FIG. 12, FIG. 13, FIG. 14). The resulting concentration is then multiplied by the dilution of the test sample to obtain the absolute concentration of the epitope-specific antibodies therein needed to achieve 50% inhibition of the respective benchmark antibody. The ACE assay data may be used to generate an antibody profile for each given test sample (see, e.g., serological profiles of FIG. 15, FIG. 16, and FIG. 17).

As provided herein, ACE assays enable: (1) the simultaneous profiling of serological responses regarding antibody specificity and quality; (2) the ability to test closely related antigens without the risk of antigenic competition; (3) a highly sensitive assay with low inter- and intra-assay variability assay; and (4) a species-independent determinations.

ACE assays may be used to monitor the development and maturation of protective responses in a subject during the course of vaccination and/or the development of a subject's acquired immunity via infection by a given pathogen.

The serological profiles obtained using an ACE assay for a given pathogen may be used to design and develop new vaccines against the given pathogen, which new vaccines may provide better protection than that conferred by existing vaccines or acquired immunity via prior infection.

As disclosed herein, monoclonal antibodies (mAbs) against epitopes of the circumsporozoite protein (CSP) of Plasmodium falciparum and sera from subjects who were immunized against Plasmodium falciparum with RTS,S vaccines are used herein to exemplify ACE assays. Particularly, “RTS,S-immune sera”, i.e., sera from subjects immunized with an RTS,S vaccine who were then subjected to a controlled human malaria infection (CHMI), and well-characterized monoclonal antibodies against several epitopes against the CSP molecule were used as the benchmark antibodies.

While the open architecture of the Mesoscale Discovery Inc. U-PLEX immunoassay platform was used in the experiments herein, any assay platform that allows the detection and distinction of specific agents over others may be employed. The U-PLEX platform is based on MSD-linkers that bind to biotinylated antigens and, when added to the assay plate, can only bind to its specific spot in the assay well. After completing the assay, the plate is inserted into the specialized reader and an electric pulse activates the luminescence of the sulfo-tagged benchmark antibodies. The resulting luminescence signal is quantified for each specific spot and the presence and amount of the signal of a given spot is indicative of which antibody outcompeted the other. For example, the amount of the signal is directly proportional to the amount that the benchmark antibody outcompeted the antibodies in the test sample and also provides an indication of the specificity and avidity of the antibodies in the test sample. Thus, a lack of a signal or weak signal indicates that antibodies in the test sample have at least a stronger binding affinity to the given epitope relative to the benchmark antibody against the same given epitope. ACE assays also allow multiplexing with other antibodies and binding fragments thereof and the cognate ligands.

The results herein indicate that ACE assays provide quantitative epitope-specificity profiles of antibody responses that may be used to differentiate between protected vs. unprotected subjects (or subjects with reduced or limited protection) against a given pathogen. As provided herein, vaccine efficacy against a given pathogen aligned with antibody specificity and affinity for epitopes spanning a given antigenic molecule of the given pathogen evidences the importance of inducing antibodies with sufficient specificity and affinity for specific epitopes for conferring protective immunity against the given pathogen. Thus, ACE assays may be used to evaluate a subject's immunity against a given pathogen. ACE assays may also be used in the research and development of vaccines, including vaccine formulations and adjuvant selections.

In the experiments herein, the particular ACE assay was designed using, as the benchmark antibodies, monoclonal antibodies that are known in the art with their specifically and affinity for specific epitopes within the CSP molecule having been determined and well-characterized. Again, as noted above, it is important to note that, with the ACE assays, any antibody in which its specificity and binding affinity for the given target epitope of interest may be employed as a benchmark antibody. That is, an ACE assay according to the present invention may be applied to any pathogen of interest so long as one has at least one antibody that specifically binds a given target epitope of the given pathogen of interest and the specificity and binding affinity/avidity to the given target epitope is known. One need not know the specific sequence of the CDRs of the antibody used as a benchmark antibody; they need only know its binding affinity/avidity to the selected target epitope that it specifically binds. Thus, in some embodiments, one or more benchmark antibodies to be used in an ACE assay may be made using methods in the art. Then the antibodies may be characterized, i.e., their binding affinity/avidity and degree of protective immunity they confer, may be determined using methods in the art. The characterized antibodies may then be used as benchmark antibodies in an ACE assay.

To exemplify an ACE assay, the antigenic epitopes and benchmark antibodies provided in Table 1 were employed:

TABLE 1

| MSD | Plate | | benchmark |
|---|---|---|---|
| Spot | Antigen | Target Epitope Sequence | antibody |
| 1 | N- terminus | GSSSNTRVLNELNYDNAGTNLYNELEMNYYGK | 5D5^{a} |
| | | QENWYSLKKNSRSLGENDDGNNEDNEKLRKP | |
| | | KHKKLKQPADG (SEQ ID NO: 1) | |
| 2 | Junction | KQPADGNPDPNANPNVDP (SEQ ID NO: 2) | CIS43^{b} |
| 3 | Minor repeats | NVDPNANPNVDPNANPNVDPNANP (SEQ ID | L9^{b} |
| | (mR) | NO: 3) | |
| 8 | Major repeats | NPNANPNANPNANPNANPNANPNA (SEQ ID | 317^{b}, 311ª |
| | | NO: 4) | |
| 9 | C-term 3D7 | HNMPNDPNRNVDENANANSAVKNNNNEEPSD | 236ª, 369^{a} |
| | | KHIKEYLNKIQNSLSTEWSPCSVTCGNGIQVRIK | |
| | | PGSANKPKDELDYANDIEKKICKMEKCS (SEQ | |
| | | ID NO: 5) | |
| 10 | C-term H18 | mid-Hamming distance = 5 | 1512ª, 369ª |
| | | HNMPNDPNRNVDENANANNAVKNNNNEEPSD | |
| | | KHIKEYLNKIQNSISTEWSPCSVTCGNGIQVRIK | |
| | | PGSADKPKDQLDYINDIEKKICKMEKCS (SEQ ID | |
| | | NO: 6) | |
| 7 | C-term H50 | most divergent from 3D7 | 1512ª, 369ª |
| | | Hamming distance = 10 | |
| | | HNMPNDPNRNVDENANANNAVKNNNNEEPSD | |
| | | KHIEQYLKTIKNSLSTEWSPCSVTCGNGIQVRIK | |
| | | PGSAGKSKNELDYENDIEKKICKMEKCS (SEQ | |
| | | ID NO: 7) | |

^{a}See Beutler, et al. (2022)
^{b}See Wang, et al. (2021)
“Major repeats” is also referred to herein as “NANP” or “CR”

To demonstrate the power of ACE assays in discerning qualitative differences and assessing antibody equivalency, RTS,S-immune serum samples from an RTS,S vaccine clinical trial (NCT00075049) were tested. The RTS,S-immune serum samples were from subjects in which their protective status against Plasmodium falciparum was known. Serological profiles of the protective responses that were induced by RTS,S vaccination were generated as described herein.

The results of the ACE assay using the RTS,S-immune sera and benchmark antibodies against CSP epitopes indicate that ACE assays are capable of assessing the equivalency of vaccine-induced antibodies in relation to benchmark antibodies against key epitopes of CSP. In this assay, the equivalence, i.e., the ability of CSP vaccine-induced antibodies to successfully compete with the benchmark antibodies for binding to their epitopes, indicates the epitope specificity, avidity, and concentration of the sera antibodies (obtained via vaccination or acquired immunity from infection) in a subject that will likely provide protective immunity against Plasmodium falciparum. That is, antibody “equivalence” herein means that the functional activity (binding and affinity) of the antibodies in a test sample are comparable or superior to the given benchmark antibodies. When benchmark antibodies are chosen based on their known functional activity against a given pathogen or antigen of interest, equivalence as determined via an ACE assay will forecast the functional activity, e.g., protective immunity, conferred by the antibodies in the test sample.

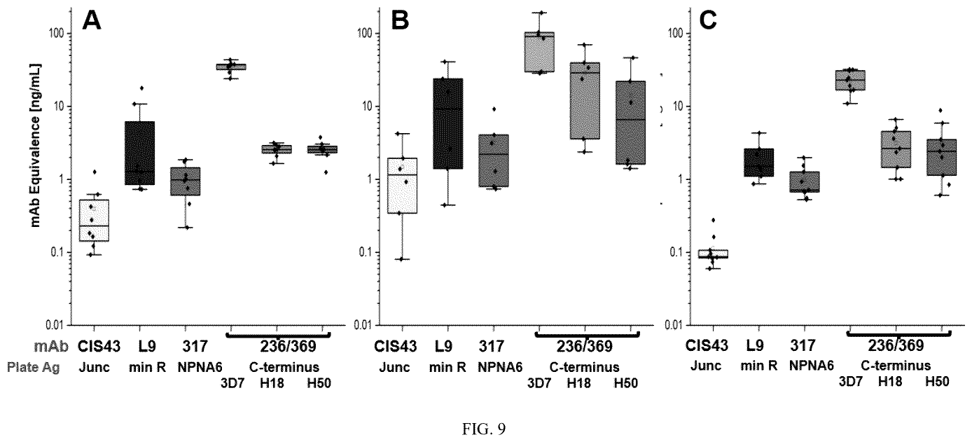

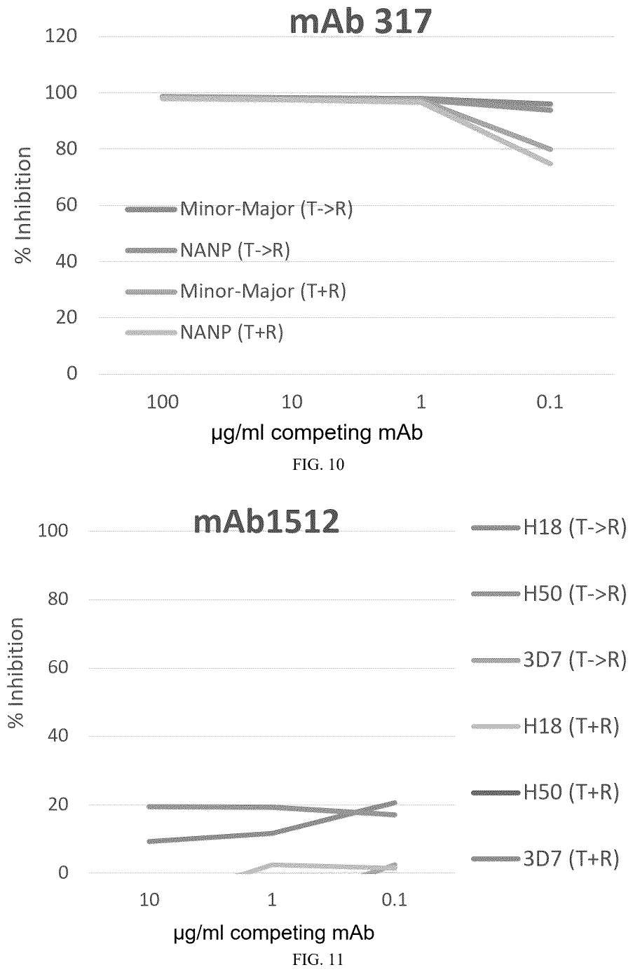

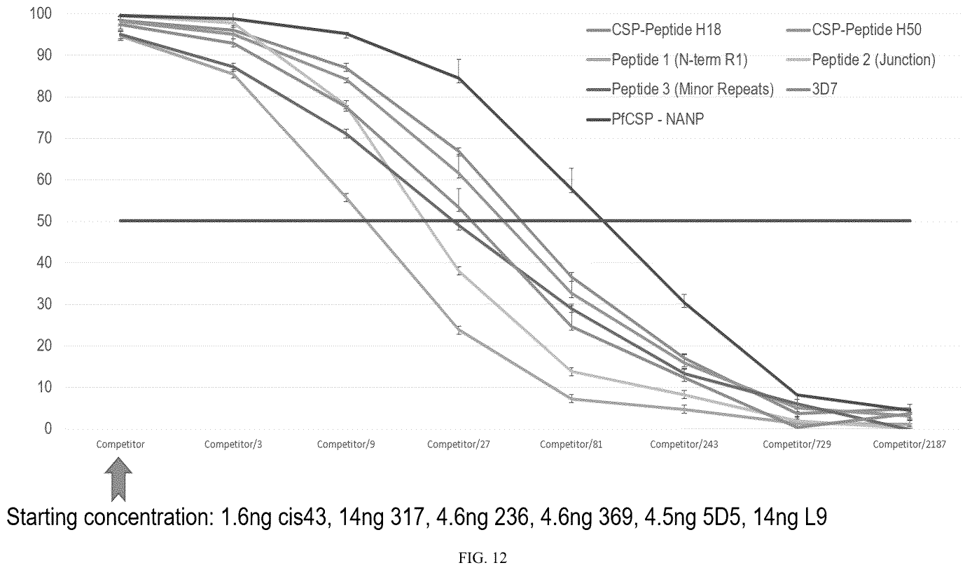

One of the key advantages of ACE assays over prior art assays for evaluating antibodies in test samples is the ability to evaluate the test samples in an unbiased manner using different, species-specific secondary antibodies. As shown in FIG. 9, ACE assays allow the testing of samples from different species (e.g., mouse, non-human primate, and human) in the same assay experiment since no species-specific reagents are needed. This enables direct qualitative and quantitative comparisons between animal models and human samples which is invaluable for (1) assessing the value of a given animal model in forecasting human responses; (2) determining differences in immunodominance of epitopes between different species and therefore prevent over-interpretation of preclinical results which, in the past, contributed to failed clinical trials. The data in FIG. 9 show that humans mount only a weak immune response to the mAb CIS43 epitope (0.1 ng/mL) while NHP have on average a 10-fold higher response (1 ng/mL). Similarly, antibody responses in NHP were 6- to 10-fold higher to the C-terminus (3D7, H18, H50) compared to human responses.

Employing ACE assays with an electro-chemiluminescence immune assay (ECLIA)-based multiplex platform provides the following advantages: (1) high sensitivity with exceptionally low inter- and intra-assay variability; (2) wide linear range over 4-5 logs; and (3) suitability for testing closely related antigens without cross reactivity due to antigenic similarity and competition; and (4) a proven suitability for samples from human vaccinated subjects. Nevertheless, any assay platform that enables the immobilization of a specific capture reagents on the assay substrate and then the detection and identification of a particular antibody bound thereto may be used, e.g., DNA barcode chips and assays wherein one or more capture reagents are individually immobilized on a substrate and identified by a unique DNA barcode. A suitable alternative to the MSD assay platform described herein, are assay platforms that employ different single-strand DNA linkers to their own given location in a assay well and then the specific capture reagent that is to be immobilized at position 1 of the assay spot has attached thereto a single-strand DNA having a sequence that is complementary to the one unique single-stranded DNA that is immobilized at position 1. Single-stranded DNA may attached to peptides (e.g., antigenic epitopes) using methods in the art.

ACE assays may be used to create a serological profile that indicates the hierarchy of antibody specificity and affinity against the antigenic epitopes of a given pathogen that provide the best protective immunity against the given pathogen. ACE assays may be used to create such serological profiles for any pathogen of interest, e.g., any pathogens belonging to viral, bacterial, protozoan, and fungal pathogen classes. In addition, ACE assays may be used to evaluate therapeutic antibodies or antibodies induced by vaccines against non-infectious diseases (e.g., Alzheimer disease), immune-mediated diseases (e.g., allergies), and other conditions (e.g., overexposure to a given substance (e.g., drugs)) that may be treated with therapeutic antibodies and/or vaccine-induced antibodies.

The experiments herein show that ACE assays simultaneously provide a serological profile of antigen specificities across a given antigenic target and measures the quality of a subject's immune response by determining the concentration of antibodies with equivalence to one or more benchmark antibodies that are functionally protective against pathogens having the given antigenic target.

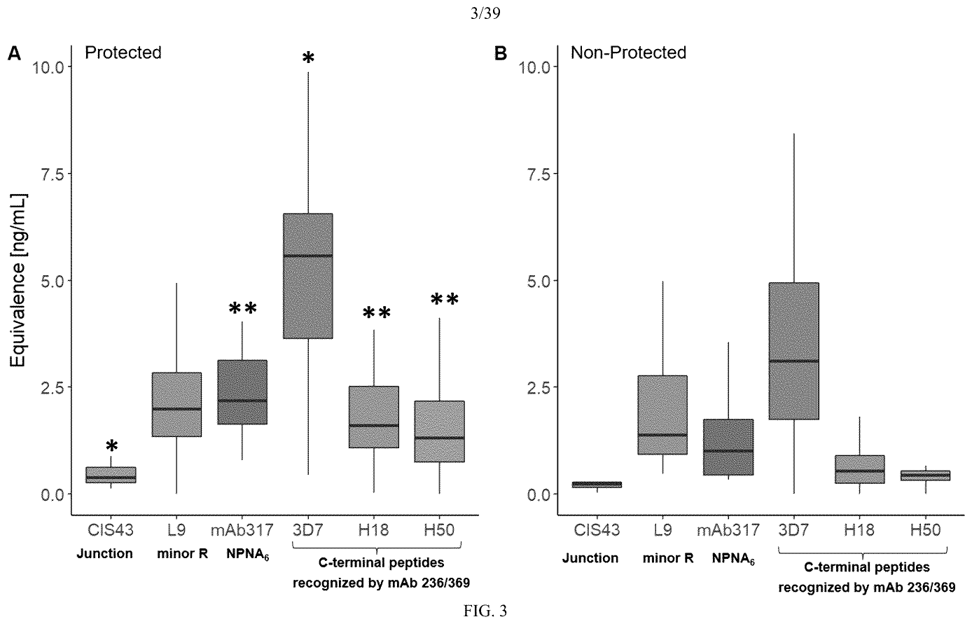

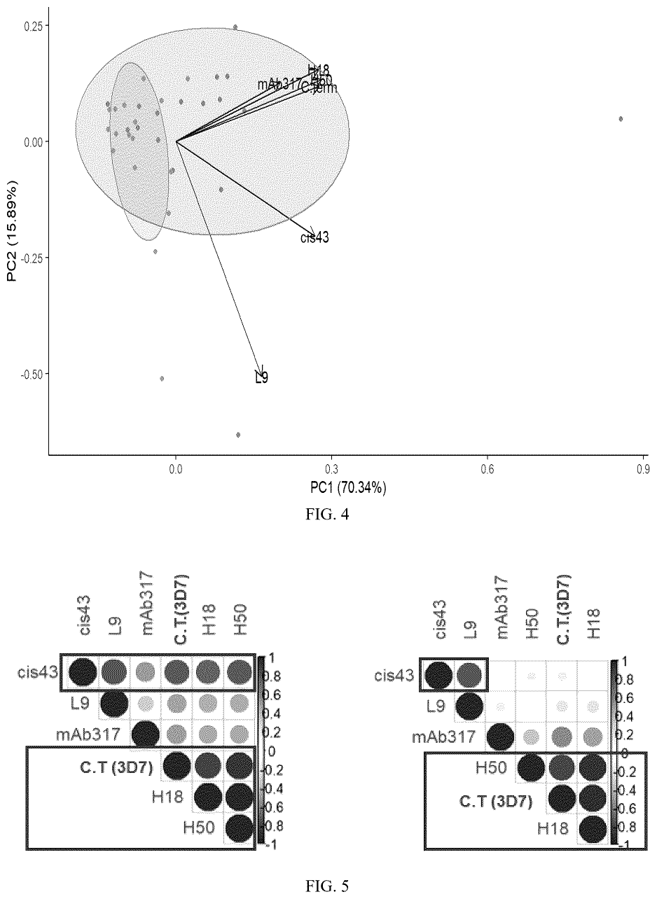

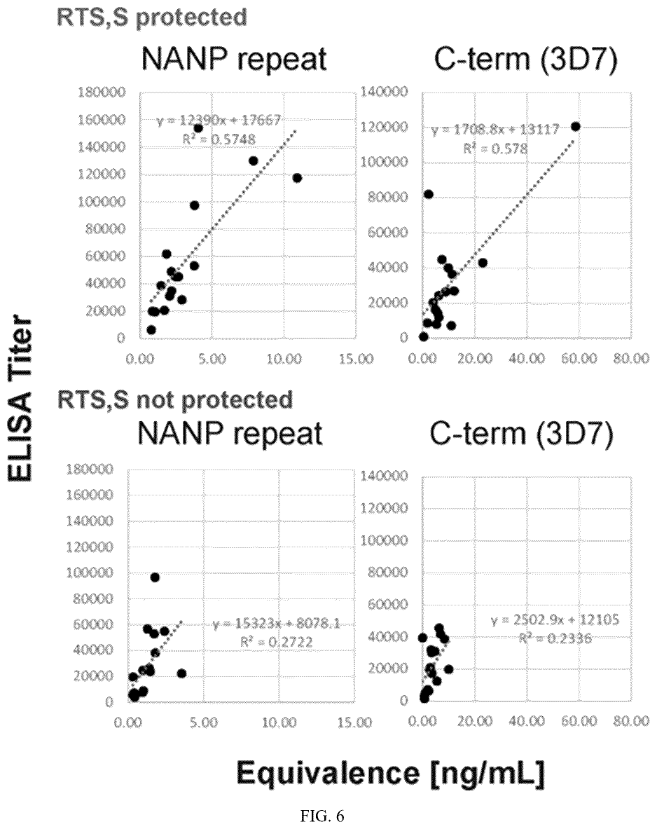

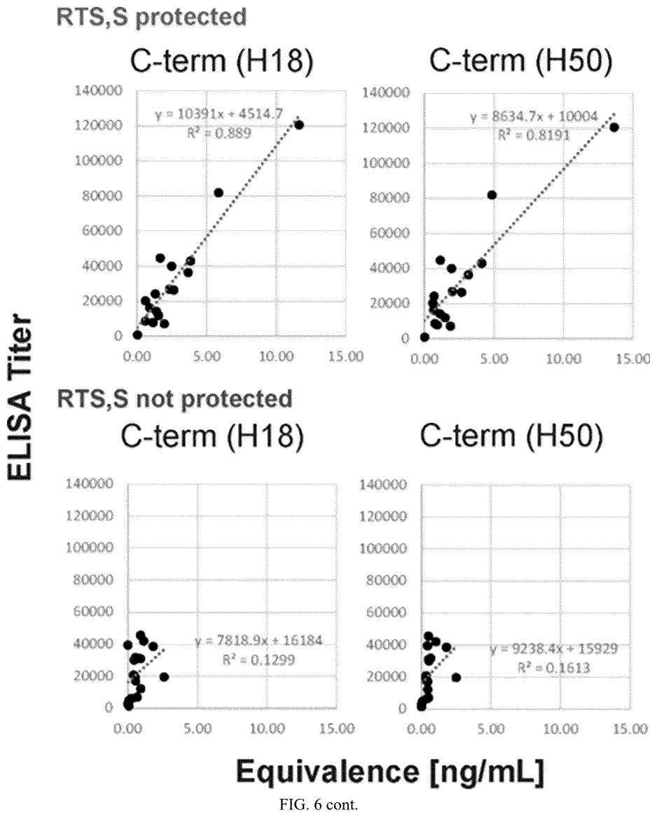

As exemplified herein:
(a) ACE assays are sensitive and highly robust assay platforms that enable the assessment of small (e.g., 0.5-10 μl) depending on the specific antibody titer in the sample volume samples. The high-throughput and high-dimensional assessment of sera is conducive to testing samples from large subject populations, e.g., clinical trials, which provide statistical sample sizes adequate for determining the functional role of specific epitopes of interest.
(b) ACE assays are capable of establishing functional antibody profiles of test samples (e.g., human sera test samples) (see, e.g., FIG. 3, FIG. 5, FIG. 15, FIG. 16, FIG. 17). This is invaluable as immunodominance may be different across particular subject populations and species and variants of a given pathogen and therefore may influence the induction and production of protective antibodies in vitro and/or in vivo in a given subject. Conventional serological assays such as ELISA and bead-based flow cytometry (e.g., LUMINEX) used to determine the quality and quantity of antibodies rely on species-specific secondary antibodies. Therefore, direct inter-species comparisons are confounded by the use of different, species-specific secondary reagents. This also inhibits inter-laboratory comparisons as different reagents such as different lots of the same secondary antibody can detrimentally affect the overall results.
(c) ACE assays provide superior results over established ELISA-based methods for assessing the magnitude of serological responses by comparison to benchmark antibodies, e.g., CSP-monoclonal, repeat-specific mAbs. ACE assays assess serological responses in a species-independent manner and provides the results as mass concentration. While prior art CSP serological assays have measured the magnitude of antibody responses, they fail to sufficiently correlate the detected antibody responses to the amount or quality of protective immunity against Plasmodium spp., e.g., P. falciparum.
(d) ACE assays may be used to measure the absolute concentration of epitope-specific antibodies in serum/plasma samples of subjects and simultaneously indicate the binding strength (avidity) of the antibody responses via competition with benchmark antibodies. The advantages of ACE assays are apparent when comparing the data with ELISA results (FIG. 6). ELISA titers to the central repeat region have been reported as an immune correlate of protection. As shown herein, ACE assays show a significantly higher concentration of major repeat-directed antibodies in protected subjects (FIG. 3), but antibodies to the minor repeats were not significantly higher in that cohort. The correlation between ELISA and ACE assay results is strong in protected individuals (r≥0.74) but lower for non-protected individuals suggesting qualitative differences in the serological responses of subjects. Qualitative differences between protected and non-protected vaccinees become even more apparent when comparing ELISA vs. ACE assay results for C-terminal antibody responses and when comparing the profiles (e.g., when establishing the breadth of an immune response to the epitopes across an entire antigen, e.g., the entire CSP molecule) (FIG. 3).

The overall agreement between ELISA and ACE assay results was primarily observed in protected RTS,S vaccinees (Table 2; FIG. 6). One possible explanation is the impact of avidity on the ACE assay results. The assay measures epitope-specific antibodies that are capable of successfully competing with the given benchmark antibodies. Successful competition of the antibodies in the test samples with the epitope specific benchmark antibodies is a function of antibody concentration, avidity, and affinity. Low-avidity antibodies may not be able to compete with high-affinity and/or high-avidity control in ACE assays but, in ELISA assays, may suggest high serum antibody titers which may deceivingly suggest immunity against the given pathogen.

One of the key differences between ELISA and ACE assays is the need by ELISAs for secondary reagents that may bias the results due to their isotype- and subclass-specificity. The results of ACE assays are independent of antibody isotypes and subclasses and, therefore, ACE assays may be used to provide a universal assessment of the immunity conferred by, e.g., a given vaccine, and also an individualized assessment of a given subject's serological response and hence degree of immunity against a given pathogen.

In the experiments herein, ACE assays indicate lower concentrations of antigen-specific antibodies in sera compared to quantitative ELISAs. This difference is the result of several factors: (i) most plate antigens present more than one epitope. For example, using the C-terminal peptide Pf16 in ELISA assays results in the binding of a multitude of different antibody clones. In the case of the ACE assays herein, only the bindings to mAb 236- and mAb 369-specific epitopes were measured. Therefore, the use of additional benchmark antibodies and evaluating the differences between multi-epitope assessment (ELISA) vs. a mono- or oligo-epitope assessment (ACE assay) may provide additional information that may be beneficial in fine-tuning ACE assays specific to protection against Plasmodium spp., e.g., P. falciparum. Measuring epitope-specific responses rather than the global response to a plate antigen may also reveal associations between epitope and protection. Another advantage of ACE assays is that the results do not only reflect the magnitude of an epitope specific response, but the results also provide a qualitative assessment, e.g., whether the avidity/affinity of a subject's serum antibodies sufficiently compete with the benchmark antibodies and thereby are understood to provide sufficient protective immunity against a given pathogen.

In a previous study, the breadth of C-terminus binding was indicated to be a surrogate marker for reactivity to conserved epitopes within the C-terminus of the CSP molecule. The ACE assays herein, which use the same set of clinical samples as the previous study, confirm that breadth as measured by the concentration of antibodies that can cross-react with a subset of C-terminus variants represented by H18 and H50 is also associated with protection (FIG. 3, FIG. 4, FIG. 5).

### Kits

Kits for performing ACE assays are also contemplated herein. The kits may comprise a plurality of antigenic epitopes of a given antigen, which each epitope is immobilized in an assigned location in each assay well of an assay substrate whereby each single assay well contains the plurality of antigenic epitopes immobilized at their own assigned location. Alternatively, the kits may comprise the plurality of antigenic epitopes with reagents, e.g., linkers, for immobilizing each epitope in the plurality in each single assay well at an assigned location on an assay substrate whereby each single assay well contains the plurality of antigenic epitopes immobilized at their own assigned location. In some embodiments, the plurality of antigenic epitopes are of a CSP molecule of a Plasmodium sp. such as P. falciparum. In some embodiments, the kits further comprise a mixture of benchmark antibodies, wherein the mixture comprises at least one benchmark antibody for each antigenic epitope in the plurality of epitopes. That is, for each antigenic epitope in the plurality of epitopes, there is at least one antibody that is capable of binding it with high affinity to form an epitope-antibody conjugate. In some embodiments, the kits further comprise a reference (which may be one or more reference samples) as described herein. In some embodiments, the kits comprise one or more reagents such as blocking buffers, assay buffers, diluents, wash solutions, benchmark antibodies, etc. In some embodiments, the kits comprise a plurality of antigenic epitopes of a given antigen packaged together with a plurality of benchmark antibodies, wherein the plurality of benchmark antibodies comprises at least one antibody that is capable of binding at least one of the epitopes with high affinity to form an epitope-antibody conjugate, whereby each epitope in the plurality of epitopes is capable of forming an epitope-antibody conjugate with at least one of the antibodies in the plurality of benchmark antibodies. In some embodiments, the kits comprise additional components such as interpretive information, control samples, reference levels, and standards.

In some embodiments, the kits include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

### Diagnostic and Prognostic Applications

The methods and kits as contemplated herein may be used in the evaluation of a subject's immunity against a given pathogen of interest. The methods and kits may be used to monitor the progress of an infection, e.g., by the active antibody response thereagainst, assess the efficacy of a vaccine, and/or identify subjects in need of being vaccinated against a given pathogen of interest (because of, e.g., lack of naturally acquired immunity by prior exposure). The methods and kits may be used to diagnose a subject as having sufficient protective immunity against a given pathogen of interest and/or provide the subject with a prognosis of overcoming the infection because of one's immune response.

In some embodiments, the methods and kits herein may be used to determine whether a subject exhibits a level of immunity against a given pathogen of interest that is low or high as compared to a reference. In some embodiments, the reference is a reference sample that is obtained from a subject who exhibits sufficient protective immunity against the given pathogen of interest. In some embodiments, the reference sample is a pooled sample from a plurality of subjects who exhibit sufficient protective immunity against the given pathogen of interest. In some embodiments, the reference is a given reference level of antibodies that should bind one or more given antigenic epitopes of the given pathogen of interest, which level of antibodies are known to provide protective immunity against the given pathogen of interest.

A subject identified as having insufficient immunity against the given pathogen of interest may be subjected to a suitable treatment, e.g., administered a vaccine against the given pathogen and/or administered one or more drugs that will help clear an infection by the given pathogen or treat the symptoms caused by the infection by the given pathogen.

In some embodiments, the methods and kits herein may be used to monitor the efficacy of a given vaccine and the vaccine regimen, e.g., timing of vaccine boosters and doses may be adjusted accordingly. Particularly, the experiments herein show the evolution of epitope-specific humoral responses in subject throughout a three-dose vaccination regimen. Thus, ACE assays may be used to evaluate whether boosters of a given vaccine only quantitatively impacts the immune response (e.g., expansion of specific antibodies) or whether one or more boosters of a given vaccine change the epitope-specific profiles in subjects, e.g., broaden and enhance the quality of the induced antibodies. That is, ACE assays may be used to determine whether one vaccine regimen provides better protection over another vaccine regimen.

### Non-Clinical Applications

In some embodiments, the methods and kits herein may be used for research purposes. For example, the methods and kits may be used to identify drugs, compounds, pharmaceutical agents, etc. that interfere with effective vaccine-induced immunity of a given vaccine against a given pathogen of interest. In some embodiments, the methods and kits may be used to identify drugs, compounds, pharmaceutical agents, adjuvants, etc. that enhance the vaccine-induced immunity of a given vaccine against a given pathogen of interest.

The data obtained from ACE assays in animals and/or humans may be used in formulating a range of dosages of given therapeutic antibodies or immunogens for use in, e.g., human subjects, and/or selecting an animal model that closely recapitulates the immune response of an intended subject, e.g., humans. The methods and kits herein may be used to develop and screen formulations to identify the optimal vaccine platform, composition and concentrations, and adjuvant for targeting protective epitopes against a given pathogen or antigen of interest and thereby develop a vaccine that will likely provide the best protection against the given pathogen or antigen. For example, the methods and kits herein may be used to profile protective serological responses in subjects and thereby determine “protective epitopes”, i.e., specific antigenic epitopes of a given pathogen or antigen of interest in which antibodies thereagainst may protective immunity. The protective epitopes and the methods and kits herein may be used to guide the selection of an appropriate animal model that similarly recapitulates the immunodominance of the respective antigen in the given subject to be treated, e.g., a human subject. A vaccine composition and concentration or amount of the one or more protective epitopes may be formulated in animal models to achieve circulating plasma concentrations of antibodies that are extrapolated to providing protective immune responses in humans.

AI-based methods, such as machine learning, may also be used in combination with the ACE assays to aid in identifying and defining serological profiles that are protective against a given pathogen or antigen of interest. For example, AI-based methods may be used to aid in determining the best candidate vaccine formulations for clinical trials.

Again, as indicated herein, ACE assays may be applied to any pathogen or antigen of interest and employed using any immunoassay platform that allows the distinction between and quantification of different epitope-antibody conjugates.

As provided herein, ACE assays provide serological equivalence information that may be expressed as concentration of serum antibodies having the same or higher avidity as compared to the given epitope specific benchmark antibodies.

The following examples are intended to illustrate but not to limit the invention.

### EXAMPLES

### Peptides and Antibodies

Seven biotinylated PfCSP peptides spanning the CSP molecule were produced (CS Bio, Menlo Park, CA), Atlantic Peptides Inc, Concord, NH)) and were used as the target antigenic epitopes. The sequences of the target epitopes are provided in Table 1. Human monoclonal antibodies that specifically bind the target epitopes, i.e., 5D5, CIS43, L9, 311, 317, 236, 369, and 1512, which are known in the art (see Beutler, et al. (2022) and Wang, et al. (2021)) were labeled using the MSD Gold-Sulfo-Tag NHS-Ester Conjugation Pack (MesoScale Discovery (MSD) Inc, Gaithersburg, MD) per the manufacturer's instructions. These sulfo-tagged mAbs were used as the benchmark antibodies in the Sulfo-Tag Reporter Mix used in the ACE assays exemplified herein. Each benchmark antibody is provided in Table 1 next to the target epitope it specifically binds.

The “Sulfo-Tag Reporter Mix” used in the experiments herein were single-use aliquot mixtures of the sulfo-tagged benchmark antibodies, which each aliquot mixture contained: 25 ng/mL N-terminus specific mAb 5D5, 25 ng/mL junctional peptide-specific mAb CIS43 60 ng/ml minor repeat-specific mAb L9, 25 ng/mL NPNA₆-specific mAb 317, 16 ng/mL α-epitope C-term mAb 236, and, 32 ng/ml β-epitope C-term mAb 369. These single-use aliquot mixtures were stored at 4° C. until use.

The “Reference Sample” used in the experiments herein was a mixture of unlabeled (no sulfo-tag) antibodies, which contained: 4.5 ng/ml mAb 5D5, 1.6 ng/ml mAb CIS43, 14 ng/mL mAb L9, 14 ng/ml mAb 317, 4.6 ng/ml mAb 236, and 4.6 ng/ml mAb 369. This Reference Sample was tested at eight, 3-fold dilutions (from neat-1:2, 187)

### Test Samples

The use of serum samples was reviewed by the WRAIR Human Subjects Protection Branch (Protocol WRAIR #2142). Pre-immune and day of challenge sera (two weeks post third immunization from a previously conducted clinical trial (NCT00075049) in which study participants vaccinated with RTS,S adjuvanted with AS01B or AS02A (n=18 protected subjects, n=18 non-protected subjects) were tested. The study enrolled both, male and female healthy U.S. residents ages 18-45 years with no travel history to malaria-endemic countries. Preliminary experiments did not show differences between the two adjuvant cohorts in the level of their antibody responses to the variant peptides nor their avidity to repeat and C-terminal 3D7 peptide. A human CSP-immune serum pool (CSP-AV) and commercial human AB pooled serum were used as positive and negative assay controls, respectively.

### ACE Assay

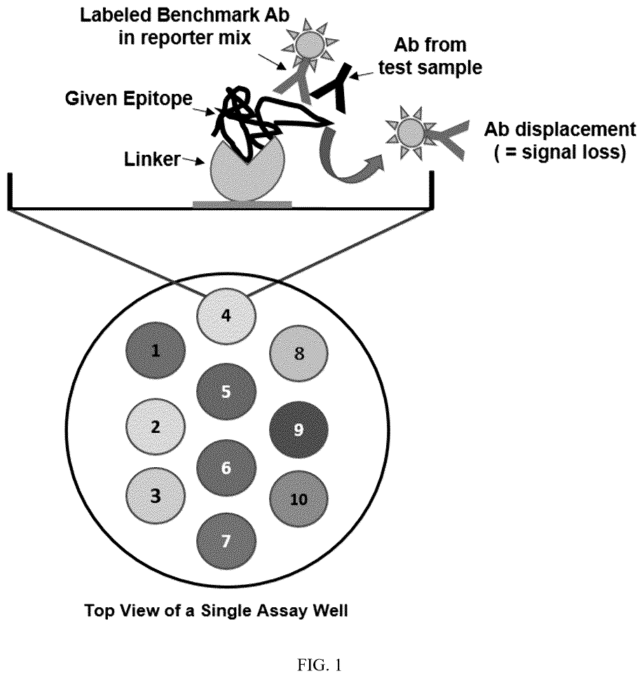

FIG. 1 schematically shows a single well of an exemplary ACE assay and the loss of signal for one of the epitopes by displacement of a labeled benchmark antibody. The ACE assay exemplified in the detailed experiments is based on the Mesoscale platform and uses the 10-spot, U-PLEX format (MSD, Gaithersburg, MD). The U-PLEX format utilizes U-PLEX linkers for targeted coating of specific spots within the wells of a U-PLEX plate. While the Mesoscale U-PLEX platform is exemplified herein, any assay platform that enables the immobilization of a specific capture reagent on an assay substrate and then the detection and identification of a particular antibody that specifically binds that specific capture reagent wherein the given capture reagent-antibody conjugate is distinguishable from other different capture reagent-antibody conjugates may be employed.

To prepare the U-PLEX assay plates, each biotinylated peptide (Table 1), representing the selected target epitopes of interest (i.e., N-terminus, junctional region, minor repeat, major repeat, and C-terminus of the CSP molecule) was diluted to 300 nM with the coating diluent (PBS with 0.5% BSA) using methods in the art. Each peptide was then mixed and incubated at room temperature (about 22-25° C.) with one of the given U-PLEX linkers provided in the MSD U-PLEX assay kit for about 30 minutes. MSD Stop Solution (MSD) was added to each peptide/linker solution (U-PLEX coupled solution) and incubated at room temperature for about 30 minutes. After incubation, the individual U-PLEX-coupled solutions were mixed together to result in a multiplex coating solution. The 10-spot U-PLEX plates were then coated with the multiplex coating solution (50 μL per well) and incubated at room temperature for 1 hour. After incubation, the U-PLEX plates were washed three times with 150 μL of 1×MSD Wash Buffer per well thereby resulting in assay wells/spots, wherein each of the given peptide/linkers are assigned, i.e., immobilized, to their own unique individualized location in the assay wells/spots. After washing with the 1×MSD Wash Buffer, the prepared U-PLEX plates were used immediately.

During the incubation of the U-PLEX multiplex coating solution, the sera samples, Sulfo-Tag Reporter Mix, Reference Sample, and dilutions thereof were prepared. The Reference Sample was tested at eight, 3-fold dilutions (from neat-1:2, 187). The sera samples were tested at four, 3-fold dilutions (1:3, 1:9, 1:27, 1:81) to generate a dose response curve for identifying the IC50 for each sera sample. Other dilutions schemes may be employed as desired. For the U-PLEX assay format, the recommended sample volume for a dilution series starts at 1:3 is 30 μL.

In a 96 well plate (Corning, Glendale, AZ), to each sera sample and Reference Sample dilution an aliquot of the Sulfo-Tag Reporter Mix was added in a 1:1 volume ratio to result in (a) test-reporter mixtures containing a given sera sample+Sulfo-Tag Reporter Mix, and (b) reference-reporter mixtures, each comprising a given dilution of a mixture of the Reference Sample+Sulfo-Tag Reporter Mix. 50 μL of each of the test-reporter mixtures and reference-reporter mixtures were added to their own given well on the antigen-coated U-PLEX plate, and incubated at room temperature for 1 hour. After incubation, the plates were washed three times with 1×MSD Wash Buffer. 150 μL of MSD Read Buffer B was added to each well and the plates were read on the MESO QuickPlex SQ 120 (MSD), per the manufacturer's instructions. Raw data was reported as mean luminescence signal (MLS).

### IC50 Determination and Quantitation of Epitope-Specific Antibody Concentrations

Test samples were diluted three-fold (1:3 to 1:81) to generate a dose response curve based on the percentage of inhibition. The dose response curve of each sample was entered into the Quest Graph™ IC50 Calculator available from AAT Bioquest on the World Wide Web at aatbio.com/tools/IC50-calculator. This calculator models an experimental data set based on a four-parameter logistic regression model and provides the dilution and the luminescence signal corresponding to the IC50. Testing eight different concentrations of the Reference Sample in each experiment was done to generate a standard curve for (a) conversion of the IC50 into absolute concentrations of epitope-specific antibodies in the sample, and (b) monitoring of assay performance from experiment to experiment. Specifically, a single-use aliquot of the Sulfo-Tag Reporter Mix was added to the Reference Sample at a 1:1 volume ratio to generate a standard curve for the quantitation of the epitope-specific antibodies. The unlabeled antibodies compete with the benchmark antibodies in the Sulfo-Tag Reporter Mix. The standard curve was then used to establish the acceptance criteria for the individual experiments since the range of inhibition should be within an inter-assay coefficient of variation (% CV)<10%.

|0112| Computational manipulations (i.e., generating the standard curve and calculating the antibody concentrations) were performed using the Standard Curve Analysis app (OriginLab Inc, Northampton, MA). For the conversion of the IC50 to a concentration of epitope-specific serum antibodies, the luminescence signal was backfitted to the standard curve. The calculated IC50 concentration was then multiplied with the dilution factor to determine the absolute concentration of epitope-specific antibody (equivalency) for each sample.

### Statistical Analysis

Univariate analysis between protected and unprotected subjects was performed to determine the correlation between MSD intensity (reactivity) to the various peptides and protection status. Prior to any t-test, a Shapiro-Wilks test was performed to determine if the to-be-compared data points were normally distributed. If both were normally distributed (p<0.05 by the Shapiro-Wilks test), a two-sided Student's t-test was applied. If either distribution was not normally distributed, the Wilcoxon signed-rank test was applied. Correlations and correlation matrices to determine the relationship between the various epitope-specificities were calculated and plotted using R Studio (Version 2023.6.1+524). Similarly, principal component analyses to identify epitopes contributing to protection (FIG. 4) were also generated in R Studio. R scripts may be obtained using methods in the art.

### Results

### Serological Profiles are Significantly Different Between Protected and Non-Protected Individuals

Establishing the serological profile of malaria-naïve, RTS,S-vaccinated subjects and stratifying the results based on protective status revealed unique roles of repeat- and C-terminal antibodies in providing protection (FIG. 3). Based on the vaccine design, the responses to the junctional sequences are presumably due to the presence of NANP/NVDP in the junctional peptide (SEQ ID NO: 2). Interestingly, the magnitude in reactivity to the minor repeat peptide was not statistically different between protected vs. non-protected individuals. In contrast, protected individuals had significantly stronger major repeat-specific (NPNA) 6 and C-terminal antibody responses compared to non-protected individuals. Also noteworthy was the fact that C-terminal antibodies of protected vaccinees demonstrated more cross-reactivity to the H18 and H50 peptides, which agrees with prior observations. No responses to the N-terminus were detected confirming the specificity of the ACE assay results since this region is not included in the RTS, S vaccine.

Correlation matrices were generated to explore the interplay between the concentrations of epitope-specific antibodies in sera of protected vs. non-protected RTS,S vaccinees (FIG. 5). In protected individuals, significant correlations were observed between their sera antibodies and the C-terminal specific control mAbs (3D7, H18, H50) (FIG. 5, left). Weaker correlations were observed for repeat-specific antibodies with only mAbs L9 and CIS43 correlating significantly. Notably, the magnitude of responses to the C-terminal peptides correlated strongly with the magnitude to the junctional region (CIS43 epitope). Taken together, the data suggest that a broad immune response to various epitopes on CSP is associated with protective immunity. Protected subjects have strong responses against the junctional region (recognized by mAb CIS43) and C-terminal responses (epitopes H18, H50, 3D7). One contributing factor to the stronger correlations in protected individuals may also be the presence of high-quality antibodies (i.e., antibodies having relatively stronger affinity and/or avidity). Such high-quality antibodies are more successful in competing with the benchmark antibodies than antibodies with relatively lower affinity and/or avidity. In contrast, in non-protected subjects, the breadth of antibody responses to the same epitopes of CSP were limited and inconsistent (FIG. 5, right). Specifically, test samples from unprotected subjects lacked antibodies that equally competed with the benchmark antibodies in binding the same CSP epitopes as did the antibodies of protected subjects and the antibody profiles were inconsistent within the group of unprotected subjects.

### Agreement Between ELISA and ACE Results

Next, the ACE assay data was compared with previously generated ELISA data that assessed total IgG, using the same plate antigens for human RTS,S vaccinees to determine the level of agreement between their results. First, the correlation between ELISA and ACE assays was determined on the entire sample set stratified by protective status (Table 2).

TABLE 2
Correlation between ELISA and ACE results

| | NPNA₆ | C-term(3D7) | H18 | H50 |
|---|---|---|---|---|
| All Subjects | 0.75 | 0.73 | 0.85 | 0.83 |
| Protected | 0.76 | 0.76 | 0.94 | 0.91 |
| Non-protected | 0.52 | 0.48 | 0.36 | 0.40 |

Overall, the correlations between the data sets are strong when analyzing the data of all subjects (r≥0.73) even though the ACE assay detects only the selected epitopes across the CSP molecule as provided in Table 1. Stratifying the data based on protective status, however, reveal a qualitative difference between the serological profiles of protected vs. non-protected individuals. The degree of correlation was lower in non-protected individuals for both the repeat and the C-terminal region. This difference was visualized by generating scatterplots for each of the plate antigens (FIG. 6).

Performance and Specificity of Region Specific mAbs

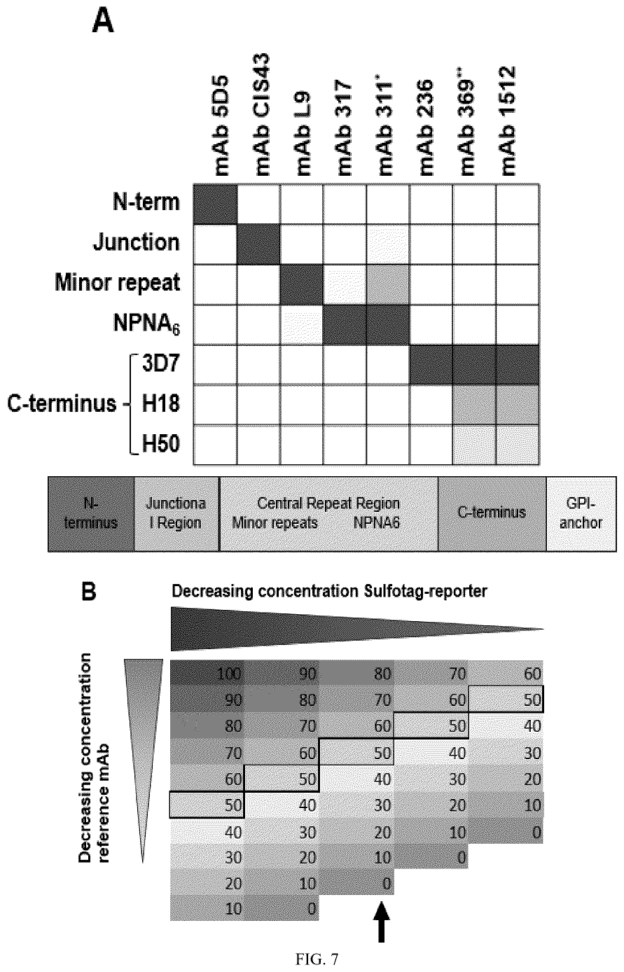

The selected human monoclonal antibodies were previously characterized in assay platforms distinct from the electro-chemiluminescence-based MSD technology used for the ACE assays herein and differences in specificity due to the high sensitivity of the assay were assessed (FIG. 7, panel A). To this end, mAbs 5D5 and CIS43 were tested against the N-terminal and the junctional peptide, the repeat specific mAbs L9, 311, and 317 were tested against junctional, the minor repeat, and the major repeat peptide. The C-terminal mAbs 236, 369, and 1512 were tested against the three C-terminal peptides (i.e., 3D7, H18, H50).

The results demonstrated high specificity for mAb 5D5 for the N-terminal peptide and no cross-reactivity to the junctional peptide. Similarly, mAb CIS43 only reacted with the junctional peptide but not with the N-terminal peptide. The reactivity pattern of major repeat (NPNA₆) detecting mAb 311 was too broad, i.e., recognized the junctional peptide, minor repeat, and major repeat, so mAb 311 was excluded from the test panel. mAb L9 reacted specifically with the minor repeat peptide while mAb 317 showed high selectivity for the major repeat, at lower concentrations, thus demonstrating their utility in the test panel.

The C-terminal mAbs 236, 369, and 1512 showed unique reaction patterns: mAb 236 reacted strongly with the 3D7 sequence of the C-terminus with no cross-reactivity to the H18 and H50 C-term haplotypes, while mAb 369 had the highest level of cross-reactivity between the three variant peptides.

### Linear Detection Range

The identification of concentrations for each benchmark antibody within the Sulfo-Tag Reporter Mix was important to determine the linear detection range. Performing the assay under conditions within the linear range assured that the ACE assay was performed within a window that allowed competition and, therefore, quantitation. In contrast, concentrations above the linear range would bias against antibodies in test samples to successfully compete with the respective benchmark antibodies. Thus, matrix experiments were conducted by varying the concentrations of each benchmark antibody to identify their optimal concentrations (FIG. 7, panel B). The various sulfo-tagged benchmark antibodies were initially tested individually for the ability to compete against themselves as unlabeled mAbs, see also Table 3.

TABLE 3
Performance of the Reference Sample

| | | | | | mAbs |
|---|---|---|---|---|---|
| | mAb 5D5 | mAb CIS43 | mAb L9 | mAb 317 | 236/369 ^{#} |
| Actual conc.^{a} | 4.5 | 1.6 | 14 | 14 | 9.2 |
| Calculated conc. ^{b} | 4.48 ± 0.13 | 1.67 ± 0.02 | 14.58 ± 0.11 | 14.19 ± 0.44 | 9.42 ± 0.10 |
| Precision ^{c} | 6.20% | 0.46% | 3.39% | 0.55% | 1.52% |
| Recovery ^{d} | 99.45% | 104.20% | 104.16% | 101.33% | 102.38% |
| Accuracy ^{e} | 100.55% | 95.80% | 95.84% | 98.67% | 97.62% |

^{#} Equivalency reported here against the 3D7 sequence of the CSP C-terminus
^{a}Actual concentration of epitope specific mAb in Reference Sample as determined by protein determination. Values expressed as ng/mL
^{b} Calculated concentration by testing Reference Sample in ACE assay (unlabeled self-competition with Sulfo-Tag Reporter Mix). Values are mean ± standard deviation of three independent experiments and expressed as ng/mL
^{c} Precision expressed as percentage coefficient of variation (% CV) of three independent experiments
^{d} Recovery [%] = (calculated value/actual value) × 100
^{e} Accuracy [%] = [1 − ((calculated value − actual value)/calculated value) × 100]

These preliminary concentrations were then used to determine potential interference between the benchmark antibodies in the Sulfo-Tag Reporter Mix. Interference was investigated particularly for the C-terminal antibodies where binding epitopes mapped to specific variant sequences.

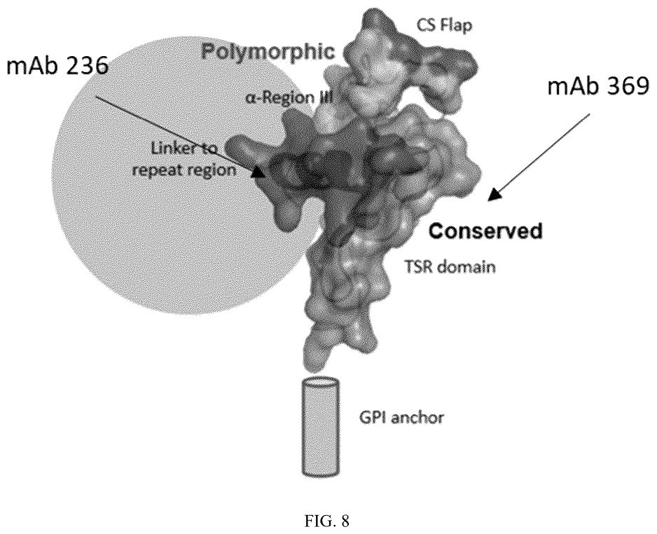

The importance of the C-terminus of CSP in mediating protective immunity is currently being investigated by various groups. The data herein suggests that reactivity of antibodies to the conserved epitopes of the β-TSR region is associated with protection in RTS, S immune individuals. Measuring the breadth of antibody responses to variant C-terminal peptides may serve as a surrogate marker for reactivity to conserved regions of the molecule and protective immunity. Here, three different C-terminal mAbs, mAbs 236, 369, and 1512, which were previously mapped to unique epitopes within the CSP molecule, were utilized (FIG. 8). mAb 236 binds to the α-helix and CS-flap of the C-terminus which is subject to polymorphisms. In contrast, mAbs 1512 and 369 bind to the conserved β-sheet regions of the C-terminus (backside), which results in the ability of the antibody to recognize the variant C-terminal peptides H18 and H50. The sulfo-tagged mAb 1512 did not bind when test samples or mAb 236 were present and was therefore excluded from further evaluation. This yet to be defined phenomenon is not present with β-sheet mAb 369. To capture the fine specificity of C-terminal antibody responses to both α- and β-epitopes, both mAbs 236 and 369 were included in the Sulfo-Tag Reporter Mix.

To assess potential interference between mAbs 236 and 369, the mAbs were tested individually and combined at two different concentrations. The mAbs did not interfere with each other but rather had an additive effect as shown in Table 4:

TABLE 4
The additive effect of mAbs 369 and 236*

| | | 236/369 mix | 236.369 |
|---|---|---|---|
| mAb 369 Single | mAb 236 Single | (actual)^{#} | (theoretical)^{$} |
| 234046 | 112063 | 361692 | 346109 |
| 134838 | 53149 | 198079 | 187987 |
| 70649 | 27505 | 104923 | 98154 |
| 38328 | 13219 | 55870 | 51547 |
| 19035 | 6900 | 27078 | 25935 |
| 9723 | 3439 | 13322 | 13162 |

*Data expressed as mean luminescence signal (MLS)
Experimentally measured MLS in multiplex assay
^{$}Arithmetically calculated luminescence signal by adding up MLS for mAbs 236 and 369 tested in single-plex assay

Thus, the final Sulfo-Tag Reporter Mix as described above was used for the matrix experiments (FIG. 7). As described above, the Reference Sample was tested in the same manner as the test samples, i.e., three-fold over eight dilutions to generate a dose response curve that is used to calculate the IC50 and then calculate the equivalency (concentration of epitope-specific antibodies). The Reference Sample was created as (a) a positive control for the ACE assay, (b) to establish inter-assay variability by bridging the assay longitudinally, (c) to serve as assay acceptance criteria. The Reference Sample was tested at lower antibody concentrations than that of the Sulfo-Tag Reporter Mix to allow for measurement of changes in the middle of the reported response. The performance of the Reference Sample was highly reproducible and the calculated concentrations closely matched the known input benchmark antibody concentrations (Table 3).

### ACE Assay Validation

A panel of sera test samples (mouse, NHP, and human) samples was generated using a particle-based CSP-vaccine (FMP014/ALFQ, NCT04296279) were tested. FMP014 is a self-assembling nanoparticle that carries (NPNA) 6 major repeat and C-terminal antigens. To demonstrate the power of ACE assays, high-throughput testing, quantitative equivalence of polyclonal antibodies induced by a CSP-vaccine formulation across preclinical models and clinical samples, and the ability to map epitope specific responses induced by vaccination were examined. The competition with well-characterized mAbs (as benchmark antibodies) provides further granularity thus allowing comparison between vaccine formulations and their ability to induce high quality responses to epitopes that are important to conferring immunity as measured by successful competition with the Sulfo-Tag Reporter Mix.

One of the unique advantages of ACE assays is an ability to directly compare the antibody profiles of test samples from subjects of different species without potential bias introduced by using secondary (anti-species) antibodies required in other assay formats (FIG. 9). The quantitation can be skewed by the quality and fine specificity of such secondary antibodies (i.e., binding heavy vs. light chain of the immunoglobulin) and the number of secondary molecules that bind to the primary antibody.

To demonstrate the power of ACE assays, serological responses induced by the FMP014 vaccine formulated with the ALFQ adjuvant were profiled. This formulation induced a strong C-terminal response in all three immunized species. However, the immune NHP sera demonstrated a stronger cross-reactivity with the variant C-terminal peptides H18 and H50 compared to humans and mice. Surprisingly, the minor repeat regions were the other focus of the immune response for all three species, despite minor repeats not being an immunogen in the FMP014 vaccine, underscoring the distinctive antibody profile compared to other vaccine formulations. Most importantly, the ACE assay was able to accurately quantify the epitope fine specificities across three species with comparable profiles observed in each species.

### Characterizing Immune Responses

ACE assays may be used to characterize immune responses induced against a given immunogen of interest (e.g., antigen or antigenic epitope thereof, vaccine, etc.).

### 1. Immune Responses Induced by Different Doses

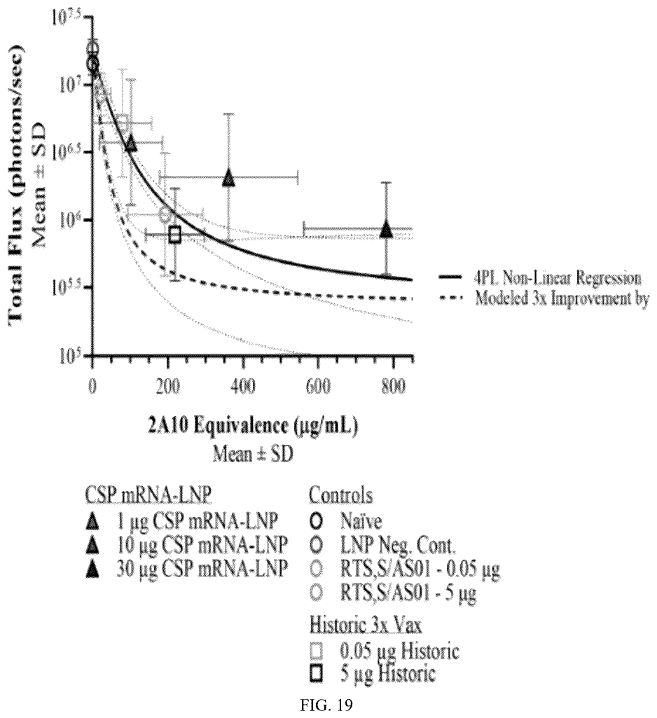

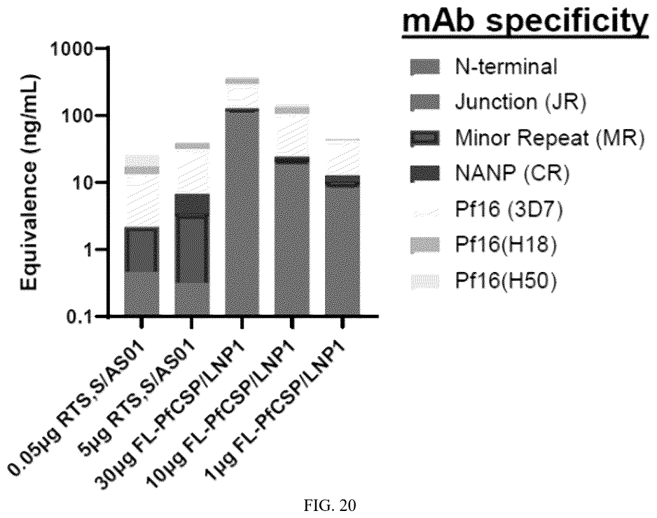

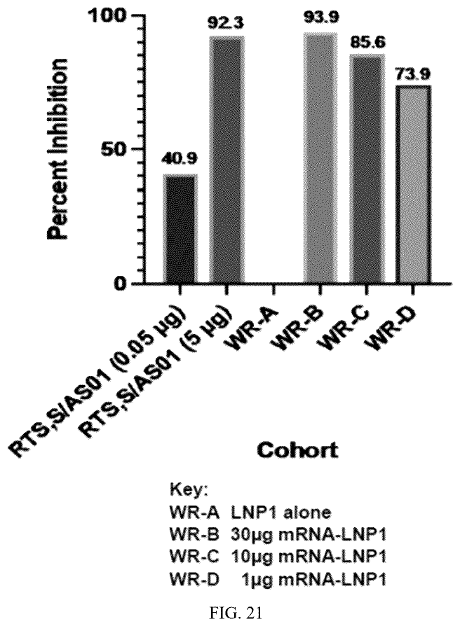

The protective immune responses induced by different doses of the “Trilink” PfCSP mRNA construct described in Mallory (2021) were examined using ACE assays. Specifically, ACE assays were performed to determine the qualitative and quantitative difference in the fine (antibody) specificities induced by the different mRNA vaccine doses. The RTS,S/ASO1B groups are reference vaccine groups. Data indicate that, in contrast to RTS,S, the mRNA construct has a unique profile as it induces immune responses to the N-terminus of the CSP protein. Mice (female, 4-6 wk-old, C57Bl/6) were divided into treatment groups and immunized with the with the given dose of the mRNA construct or control as indicated in FIG. 19. ACE assays were performed using the benchmark antibodies as described in Table 1 and the results are provided in FIG. 20. Subjects were i.v. challenged with the same amount of the 2000 P. berghei ANKA strain at 3 weeks post immunization. Percent inhibition was calculated by relating Plasmodium falciparum 18S rRNA detection in hepatocytes incubated with test sera from immune mice to a standardized measurement of parasite growth in human hepatocytes (i.e., hepatocytes incubated with control sera). Data reveals that 30 μg of the mRNA construct and 5 μg of RTS,S/AS01 provide similar inhibition percentages (FIG. 21), but via significantly different induced antibody responses (FIG. 20).

### 2. Immune Responses Induced by Different Antigens

ACE assays were used to characterize and map the fine (antibody) specificities of antibody responses induced by different subunit mRNA constructs. The different subunits of the PfCSP encoded by the mRNA constructs were:
N-term/JR/MR/CR-L-F/LNP1 (N-term, junctional/minor repeat/central repeat of PfCSP)
JR/MR/CR-L-F/LNP1 (junctional/minor repeat/central repeat of PfCSP)
JR/MR-L-F/LNP1 (junctional/minor repeat of PfCSP)
CR-L-F/LNP1 (only the central repeat of PfCSP)
PfCSP 2.0 (tr2)/KC2 (full-length CSP with regulatory elements and ionizable cationic lipid KC2, see Mendonça (2023))
FL-CSP (Trilink)/KC2 (full-length CSP and ionizable cationic lipid KC2, see Mallory (2021))

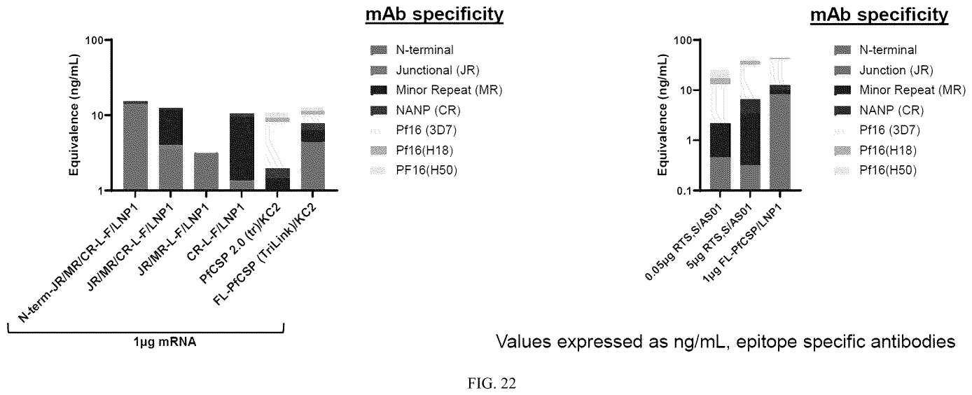

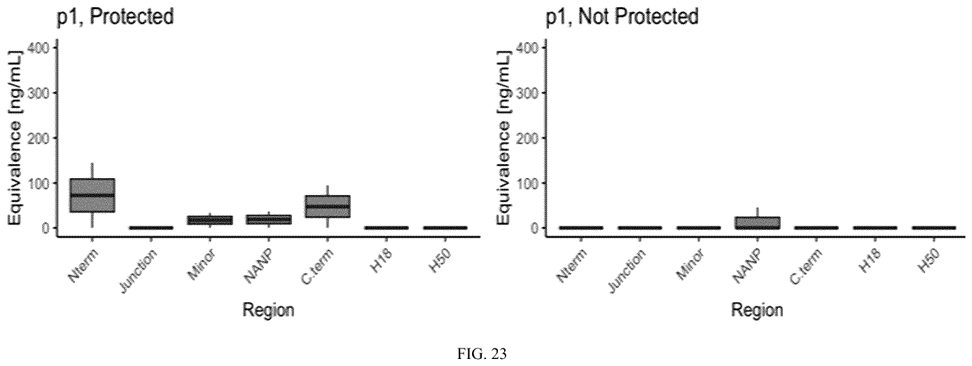

Mice (female, 4-6 wk-old, C57Bl/6 mice) were immunized with 1 μg of a given mRNA construct. FIG. 22 shows that the different subunits induce different antibody responses. The ACE assay was able to show a clear distinction between the two constructs that encode the full length PfCSP, i.e., PfCSP2.0 (tr2)/KC2 and FL-PfCSP(TriLink) KC2. The addition of the tr2 regulatory sequence resulted in a distinct antibody profile that was focused on the C-terminus (=Pf16). In contrast, FL-PfCSP(TriLink) KC2 resulted in an antibody response that was focused on the N-terminus. The subunit mRNA constructs packaged in LNP1 without KC2 only induced epitope specific responses and overcame the immunodominance of the central repeat region. These results indicate that ACE assays may be used to elucidate differences in immune responses that are caused by different antigens and/or different vaccine formulations.

ACE assays were also used to evaluate the immune responses to different Tobacco mosaic virus (TMV) capsid virus-like particles displaying major, minor and junctional circumsporozoite protein epitopes as described in Ryan, et al. (Comparison of major, minor and junctional circumsporozoite protein epitopes for malaria vaccine design. NPJ Vaccines. 2025 Oct. 3; 10 (1): 215. doi: 10.1038/s41541-025-01264-0), which is herein incorporated by reference in its entirety. The antibody profiles were then used to select the most promising TMV construct for further research and development as a vaccine candidate.

### Monitoring the Evolution of Immune Responses

Like the use of ACE assays to monitor the longitudinal changes in human antibody profiles induced by the RTS,S/AS01B (FIG. 17), ACE assays were also used to monitor the longitudinal changes in human antibody profiles induced by the FMP13/ALFQ vaccine candidate of Hutter et al. (2022).

The vaccine was delivered using three distinct regimens: Standard regimen (three immunizations at month 0, 1, and 2), delayed full dose (three immunizations with full dose at month 0, 1, 7) and delayed fractional dose (two full doses at month 0, 1, and a fractional (1/5 of full dose) dose at month 7. Protection was defined by sterile protection throughout the 28 days post challenge. Some individuals had a significant onset of parasitemia, which was scored as partial protection.

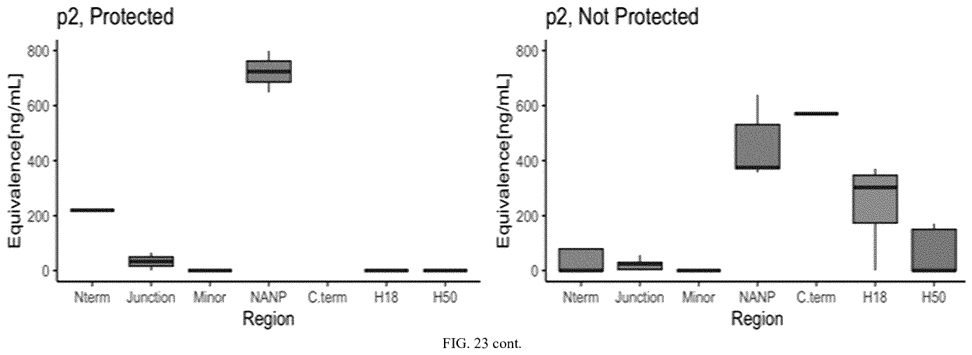

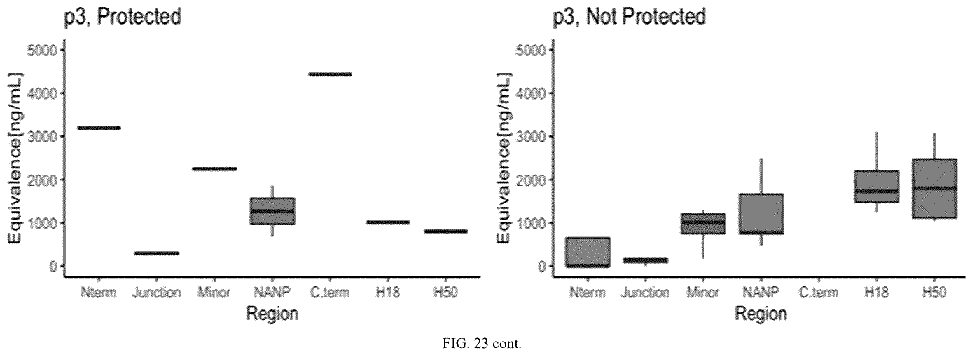

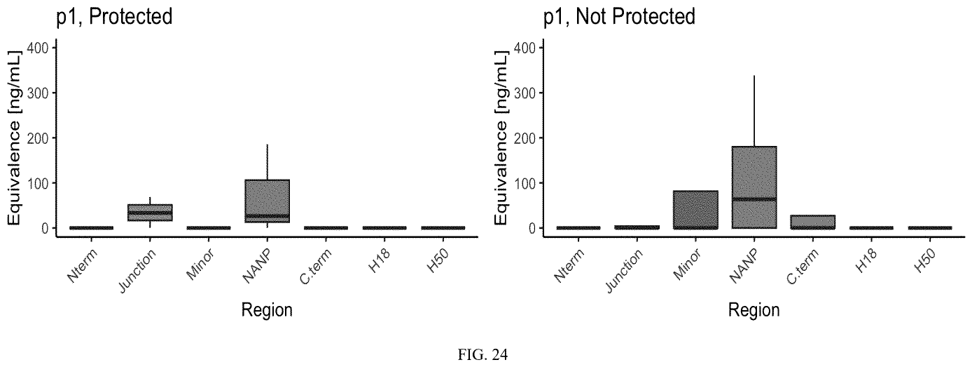

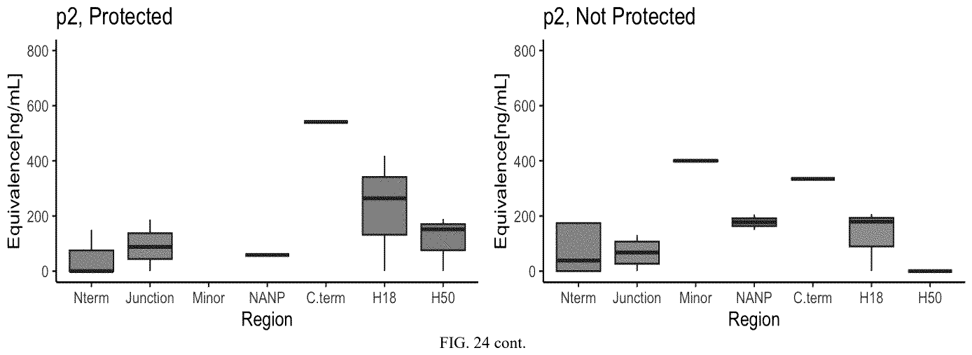

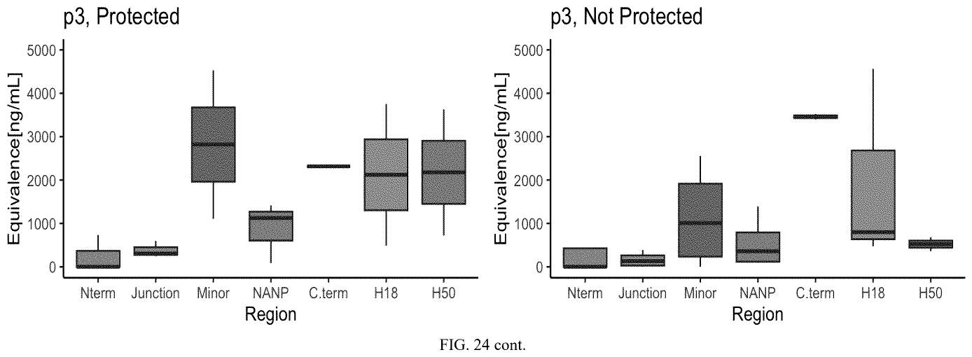

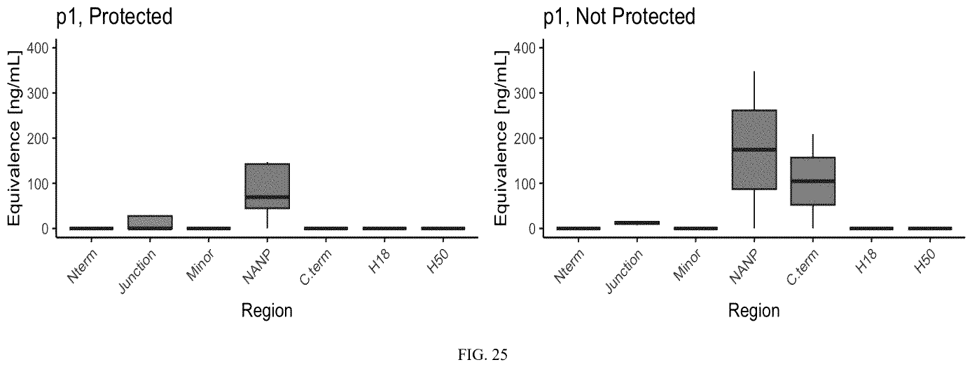

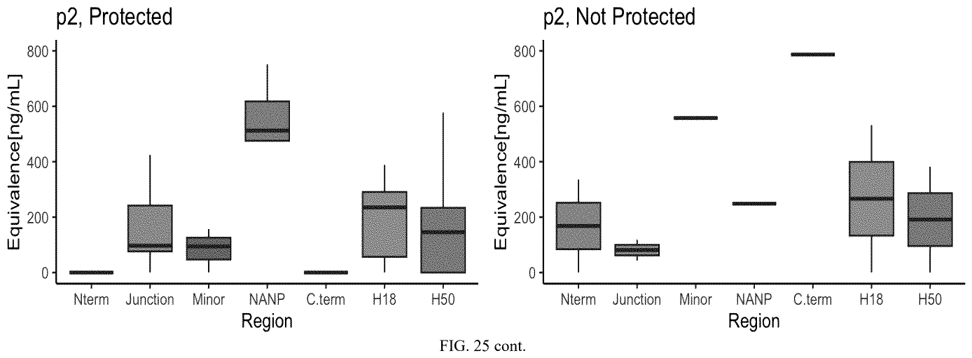

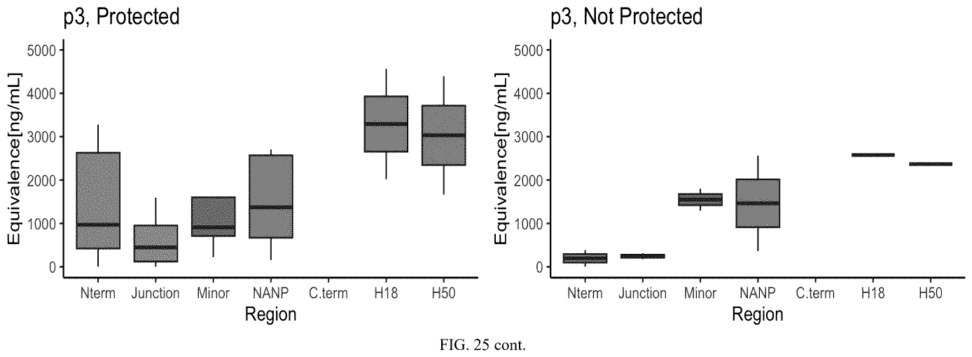

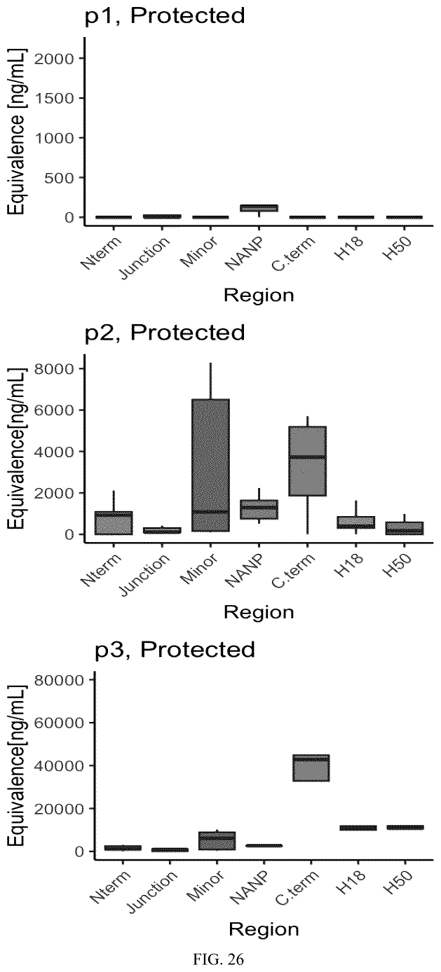

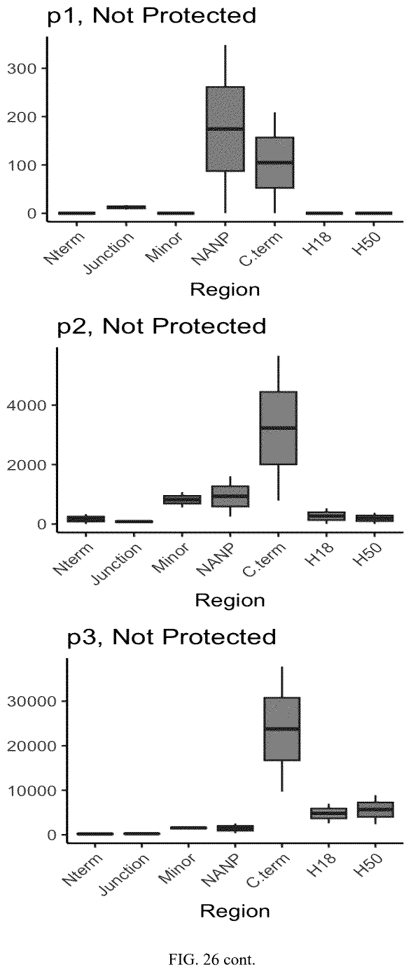

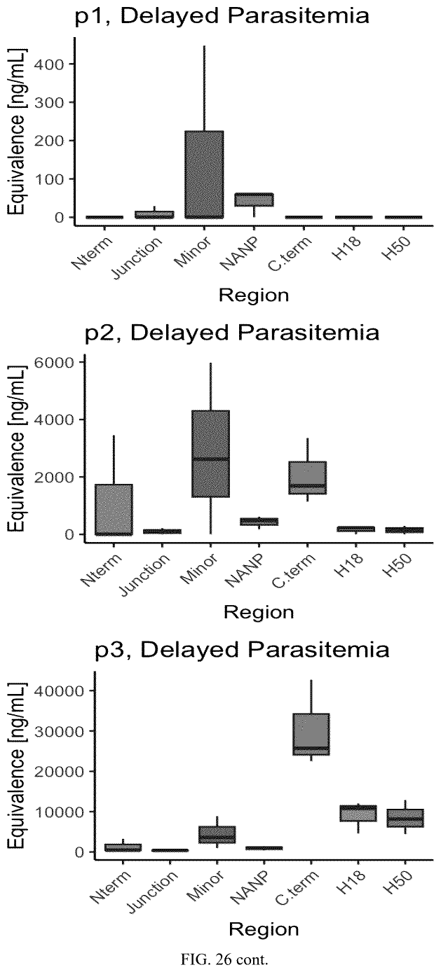

Using the ACE assays and the benchmark antibodies as described in Table 1, the evolution of immune responses, i.e., the qualitative and quantitative changes in the fine (antibody) specificities to the CSP epitopes including the N-terminus (unlike the RTS,S vaccine candidate, FMP13 contains the N-terminus). The results are provided in FIG. 23 to FIG. 26.

### Modeling Immune Responses

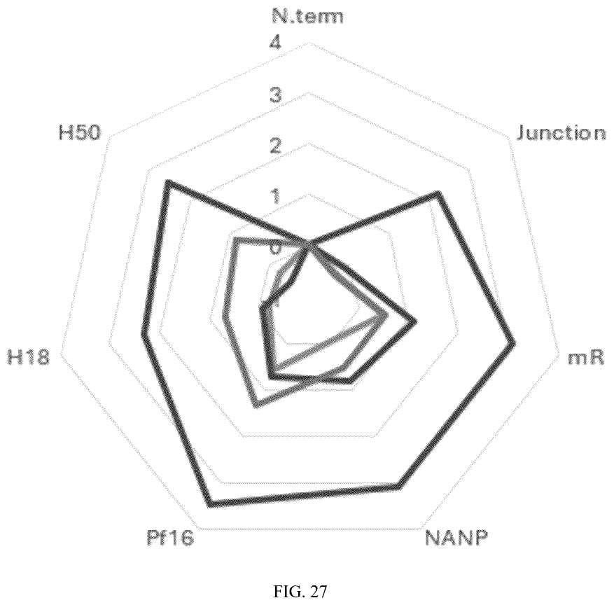

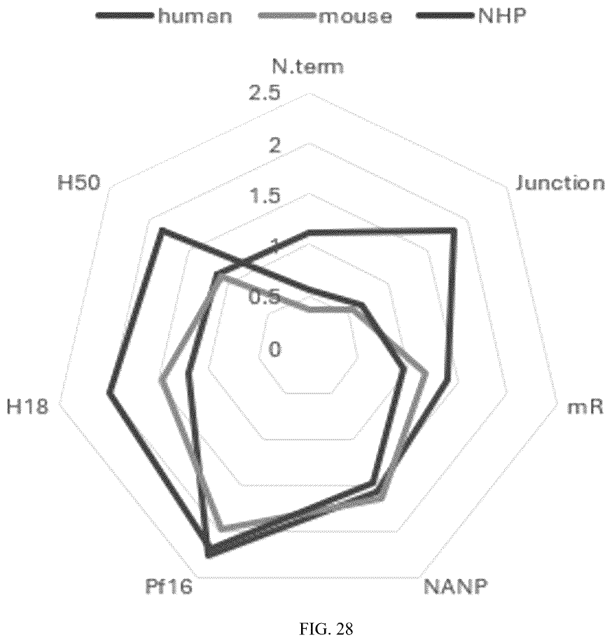

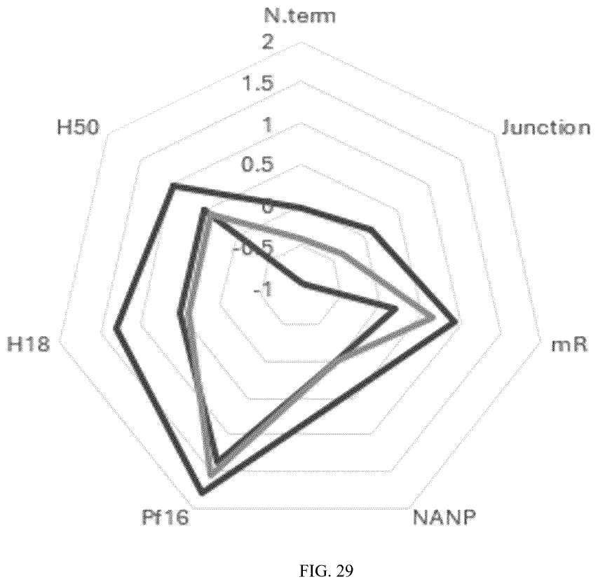

The ACE assays may be used to model and compare the immune responses in different subjects. ACE assays were used to obtain the antibody profiles in different subjects against three different CSP-based vaccines, i.e., RTS,S/AS01B (FIG. 27), FMP13/ALFQ (FIG. 28), and FMP14/ALFQ described in Seth (2017) (FIG. 29). The data shown in FIG. 27 to FIG. 29 indicate that the best animal model of the likely human immune response varies for different vaccines. Thus, ACE assays may be used to select the overall animal model for studies with a given vaccine. The ACE assays may also be used to select the best animal model for studying the antibody response against a particular antigenic epitope of interest. For example, one might select the rat as the animal model to study agents that likely interfere with the ability of the RTS,S/AS01B vaccine to induce antibodies against the mR epitope in humans, but select the mouse to study agents that likely interfere with the ability of the RTS,S/AS01B vaccine to induce antibodies against the H50 epitope in humans.

### Additional Embodiments

Embodiment 1: A universal species-independent method for characterizing unknown antibodies that may be present in a test sample, which comprises (a) selecting a plurality of epitopes of a given antigen of interest, wherein one or more epitopes in the plurality of epitopes are known to induce a protective antibody response against the given antigen in a subject when the subject is vaccinated with the one or more epitopes; (b) obtaining a reporter mixture comprising a plurality of benchmark antibodies, each capable of binding one or more epitopes in the plurality of epitopes when contacted therewith; (c) obtaining an assay substrate comprising the plurality of epitopes immobilized on a first single assay well, wherein the epitopes immobilized on the first single assay well are capable of being distinguished from each other; (d) mixing the test sample with an amount of the reporter mixture to give a test mixture; (e) contacting the plurality of epitopes immobilized on the first single assay well with the test mixture and then detecting the formation of the one or more epitope-antibody conjugates; and (f) characterizing the unknown antibodies in the test sample as being protective when the unknown antibodies equally compete with or outcompete the plurality of benchmark antibodies in the amount of the reporter mixture in forming epitope-antibody conjugates with the one or more epitopes known to induce a protective antibody response that are immobilized on the first single assay well.

Embodiment 2: The method according to Embodiment 1, wherein the concentration of each benchmark antibody in the plurality of benchmark antibodies in the reporter mixture does not exceed its linear detection range, wherein the linear detection range of a given antibody is a concentration range in which equal competition between the given antibody and itself for binding its cognate ligand is detectable. Embodiment 3: The method according to Embodiment 2, wherein the linear detection range of each benchmark antibody is determined by conducting a plurality of competition assay experiments between benchmark antibody test mixtures and at least one benchmark antibody reporter mixture, wherein the benchmark antibody test mixtures have differing concentrations of the plurality of benchmark antibodies, and wherein the benchmark antibodies in the benchmark antibody reporter mixture are distinguishable from the benchmark antibodies in the benchmark antibody test mixtures.

Embodiment 4: The method according to any one of Embodiments 1 to 3, wherein step (f) comprises comparing the formed epitope-antibody conjugates with a reference.

Embodiment 5: The method according to Embodiment 4, wherein the reference is the amounts of each benchmark antibody in the plurality of benchmark antibodies that equally compete with themselves for binding each epitope immobilized on a second single assay well, said second single assay well is a duplicate of the first single assay well.

Embodiment 6: The method according to Embodiment 4, wherein the reference is a profile of epitope-antibody conjugates formed between antibodies of a reference-reporter mixture, said reference-reporter mixture comprising (1) a reference sample having the plurality of benchmark antibodies the same amount as in the reporter mixture that was mixed with the test sample, and (2) the same amount of the reporter mixture that was mixed with the test sample, wherein, when bound to one or more epitopes immobilized on second single assay well, said second single assay well is a duplicate of the first single assay well, the benchmark antibodies from the reference sample are distinguishable from the benchmark antibodies from the reporter mixture.

Embodiment 7: The method according to Embodiment 6, which further comprises (i) contacting the reference-reporter mixture with the plurality of epitopes immobilized on the second single assay well; and (ii) comparing the amounts of the benchmark antibodies from the reporter mixture that are bound to each epitope in the plurality of epitopes immobilized in the first single assay well to the amounts of either the benchmark antibodies of the reference sample or the benchmark antibodies in the reporter mixture that are bound to the plurality of epitopes immobilized on the second single assay well.

Embodiment 8: The method according to any one of Embodiments 1 to 7, which further comprises obtaining a standard curve for each epitope in the plurality of epitopes, wherein the standard curve of a given epitope represents the amounts of the plurality of the benchmark antibodies in the reporter mixture that outcompete themselves over several different concentrations and/or dilutions of the concentration of the plurality of the benchmark antibodies present in the reporter mixture and thereby bind the given epitope that is immobilized in single assay wells that are duplicates of the first single assay well.

Embodiment 9: The method according to any one of Embodiments 1 to 8, which further comprises obtaining a dose response curve of the test sample that represents different dilutions and/or concentrations of the test sample titrated against the reporter mixture.

Embodiment 10: The method according to any one of Embodiments 1 to 9, which further comprises obtaining a dose response curve of several different concentrations and/or dilutions of a reference sample titrated against the reporter mixture, wherein said reference sample comprises the same benchmark antibodies as in the reporter mixture and in the same concentrations.

Embodiment 11: The method according to any one of Embodiments 1 to 10, wherein each benchmark antibody in the plurality of benchmark antibodies in the reporter mixture have a detectable label.

Embodiment 12: A method of obtaining an antibody profile of a subject, which comprises performing the method according to any one of Embodiments 1 to 11 on a sample obtained from the subject that is used as the test sample.

Embodiment 13: The method according to Embodiment 12, which further comprises administering to the subject a composition comprising the given antigen of interest or an antigenic epitope thereof before and/or after obtaining the sample from the subject.

Embodiment 14: The method according to Embodiment 12, which comprises challenging the subject with a pathogen that expresses the given antigen of interest before and/or after obtaining the sample from the subject.

Embodiment 15: The method according to Embodiment 13, which comprises challenging the subject with a pathogen that expresses the given antigen of interest before and/or the subject is administered the composition.

Embodiment 16: The method according to any one of Embodiments 1 to 15, wherein the plurality of epitopes is of a circumsporozoite protein (CSP) of a Plasmodium spp, e.g., Plasmodium falciparum, and, optionally, the plurality of benchmark antibodies comprise one or more of the following: mAb 5D5, mAb CIS43, mAb L9, mAb 317, mAb 311, mAb 236, mAb 1512, mAb 369.

Embodiment 17: A kit comprising (a) (1) a plurality of antigenic epitopes of a given antigen, which each antigenic epitope is immobilized in an assigned location in each assay well of an assay substrate whereby each single assay well contains the plurality of antigenic epitopes immobilized at their own assigned location; or (2) the plurality of antigenic epitopes and linkers along with reagents for immobilizing each antigenic epitope in the plurality at an assigned location in each assay well of the assay substrate; (b) a reporter mixture comprising a plurality of benchmark antibodies; and (c) a reference.

Embodiment 18: The kit according to Embodiment 17, wherein the reference is a reference sample.

Embodiment 19: The kit according to Embodiment 17 and/or Embodiment 18, wherein the plurality of antigenic epitopes is of a circumsporozoite protein (CSP) of a Plasmodium spp, e.g., Plasmodium falciparum.

Embodiment 20: The kit according to any one of Embodiments 17 to 19, wherein the plurality of benchmark antibodies comprise one or more of the following: mAb 5D5, mAb CIS43, mAb L9, mAb 317, mAb 311, mAb 236, mAb 1512, mAb 369.

### REFERENCES

The following references are herein incorporated by reference in their entirety with the exception that, should the scope and meaning of a term conflict with a definition explicitly set forth herein, the definition explicitly set forth herein controls:
Aldrich, et al. Roles of the amino terminal region and repeat region of the Plasmodium berghei circumsporozoite protein in parasite infectivity PloS One (2012) 7 (2): e32524 doi: 10.1371/journal.pone.0032524.
Beutler, et al. A novel CSP C-terminal epitope targeted by an antibody with protective activity against Plasmodium falciparum PloS Pathog (2022) 18 (3): e1010409 doi: 10.1371/journal.ppat. 1010409.
Bolton, et al. Comparison of elisa with electro-chemiluminescence technology for the qualitative and quantitative assessment of serological responses to vaccination Malar J (2020) 19 (1): 159 doi: 10.1186/s12936-020-03225-5.
Bolton, et al. Multiplex serological assay for establishing serological profiles of polymorphic, closely related peptide antigens MethodsX (2021) 8:101345 doi: 10.1016/j.mex.2021.101345.
Bolton, et al. Novel Antibody Competition Binding Assay Identifies Distinct Serological Profiles Associated With Protection Frontiers Immunol, Vol 14 doi: 10.3389/fimmu.2023.1303446.
Cairns, et al. The duration of protection against clinical malaria provided by the combination of seasonal rts,S/as01 (E) vaccination and seasonal malaria chemoprevention versus either intervention given alone BMC Med (2022) 20 (1): 352 doi: 10.1186/s12916-022-02536-5.
Cawlfield, et al. Safety, toxicity and immunogenicity of a malaria vaccine based on the circumsporozoite protein (FMP013) with the adjuvant army liposome formulation containing QS21 (ALFQ). Vaccine. 2019 Jun. 27; 37 (29): 3793-3803. doi: 10.1016/j.vaccine.2019.05.059.
Chandramohan, et al. Seasonal malaria vaccination with or without seasonal malaria chemoprevention New Engl J Med (2021) 385 (11): 1005-17 doi: 10.1056/NEJMoa2026330.
Chaudhury, et al. Assessing Prevalence and Transmission Rates of Malaria through Simultaneous Profiling of Antibody Responses against Plasmodium and Anopheles Antigens J Clin Med (2022) 11 (7): 1839 doi: 10.3390/jcm11071839.
Chaudhury, et al. Breadth of humoral immune responses to the C-terminus of the circumsporozoite protein is associated with protective efficacy induced by the rts, S malaria vaccine Vaccine (2021) 39 (6): 968-75 doi: 10.1016/j.vaccine.2020.12.055.
Chaudhury, et al. Delayed fractional dose regimen of the rts, S/as01 malaria vaccine candidate enhances an igg4 response that inhibits serum opsonophagocytosis Sci Rep (2017) 7 (1): 7998 doi: 10.1038/s41598-017-08526-5.
Chaudhury, et al. Identification of Immune Signatures of Novel Adjuvant Formulations Using Machine Learning Sci Rep, 8 (1), 17508 doi: 10.1038/s41598-018-35452-x.
Chaudhury, et al. The biological function of antibodies induced by the rts, S/as01 malaria vaccine candidate is determined by their fine specificity Malar J (2016) 15:301 doi: 10.1186/s12936-016-1348-9.
Collins, et al. Enhancing protective immunity to malaria with a highly immunogenic virus-like particle vaccine Sci Rep (2017) 7:46621 doi: 10.1038/srep46621.
Coppi, et al. The malaria circumsporozoite protein has two functional domains, each with distinct roles as sporozoites journey from mosquito to mammalian host J Exp Med (2011) 208 (2): 341-56 doi: 10.1084/jem.20101488.
Datoo, et al. Efficacy of a low-dose candidate malaria vaccine, R21 in adjuvant matrix-M, with seasonal administration to children in Burkina Faso: A randomised controlled trial Lancet (2021) 397 (10287): 1809-18 doi: 10.1016/s0140-6736 (21) 00943-0.
Dennison, et al. Magnitude, specificity, and avidity of sporozoite-specific antibodies associate with protection status and distinguish among rts, S/as01 dose regimens Open Forum Infect Dis (2020) 8 (2) doi: 10.1093/ofid/ofaa644.
Dobano, et al. Concentration and avidity of antibodies to different circumsporozoite epitopes correlate with rts, S/asOle malaria vaccine efficacy Nat Commun (2019) 10 (1): 2174 doi: 10.1038/s41467-019-10195-z.
Flores-Garcia, et al. The P. Falciparum CSP repeat region contains three distinct epitopes required for protection by antibodies in vivo PloS Pathog (2021) 17 (11): e1010042 doi: 10.1371/journal.ppat.1010042.
Gaudinski, et al. A monoclonal antibody for malaria prevention New Engl J Med (2021) 385 (9): 803-14 doi: 10.1056/NEJMoa2034031.
Hutter, et al. First-in-human assessment of safety and immunogenicity of low and high doses of Plasmodium falciparum malaria protein 013 (FMP013) administered intramuscularly with ALFQ adjuvant in healthy malaria-naïve adults. Vaccine. 2022 Sep. 22; 40 (40): 5781-5790. doi: 10.1016/j.vaccine.2022.08.048.
Imkeller, et al. Antihomotypic affinity maturation improves human B cell responses against a repetitive epitope Science (2018) 360 (6395): 1358-62 doi: 10.1126/science.aar5304.
Kester, et al. Randomized, double-blind, phase 2a trial of falciparum malaria vaccines rts, S/as01b and rts, S/as02a in malaria-naive adults: safety, efficacy, and immunologic associates of protection J Infect Dis (2009) 200:337-46 doi: 10.1086/600120.
Kisalu, et al. A human monoclonal antibody prevents malaria infection by targeting a new site of vulnerability on the parasite Nat Med (2018) 24 (4): 408-16 doi: 10.1038/nm.4512.
Laurens M B. Rts,S/as01 vaccine (Mosquirix): an overview Hum Vaccin Immunother (2020) 16 (3): 480-9 doi: 10.1080/21645515.2019.1669415.
Mallory, et al. Messenger RNA expressing PfCSP induces functional, protective immune responses against malaria in mice. NPJ Vaccines. 2021 Jun. 18; 6 (1): 84. doi: 10.1038/s41541-021-00345-0.
Martin, et al. Affinity-matured homotypic interactions induce spectrum of pfcsp-antibody structures that influence protection from malaria infection bioRxiv (2022), 2022.09.20.508747 doi: 10.1101/2022.09.20.508747.
Mendonça, et al. Design of lipid-based nanoparticles for delivery of therapeutic nucleic acids. Drug Discov Today. 2023 March; 28 (3): 103505. doi: 10.1016/j.drudis.2023.103505.
Murugan, et al. Evolution of protective human antibodies against Plasmodium falciparum circumsporozoite protein repeat motifs Nat Med (2020) 26 (7): 1135-45 doi: 10.1038/s41591-020-0881-9.
Neafsey, et al. Genetic diversity and protective efficacy of the rts, S/as01 malaria vaccine New Engl J Med (2015) 373 (21): 2025-37 doi: 10.1056/NEJMoa1505819.
Organization, W. H. (2021). Full evidence report on the RTS, S/AS01 malaria vaccine SAGE Yellow B October 2021, 1-90.
Oyen, et al. Cryo-em structure of P. Falciparum circumsporozoite protein with a vaccine-elicited antibody is stabilized by somatically mutated inter-fab contacts Sci Adv (2018) 4 (10): eaau8529 doi: 10.1126/sciadv.aau8529.
Oyen, et al. (2017). Structural basis for antibody recognition of the NANP repeats in <i>Plasmodium falciparum</i>circumsporozoite protein Proceedings of the National Academy of Sciences, 114 (48), E10438-E10445 doi: 10.1073/pnas. 1715812114.
Pholcharee, et al. Diverse antibody responses to conserved structural motifs in Plasmodium falciparum circumsporozoite protein J Mol Biol (2020) 432 (4): 1048-63 doi: 10.1016/j.jmb.2019.12.029.
Pholcharee, et al. Structural and biophysical correlation of anti-nanp antibodies with in vivo protection against P. Falciparum Nat Commun (2021) 12 (1): 1063 doi: 10.1038/s41467-021-21221-4.
Plotkin S A. Updates on immunologic correlates of vaccine-induced protection Vaccine (2020) 38 (9): 2250-7 doi: 10.1016/j.vaccine.2019.10.046.
Radin, et al. A monoclonal antibody-based immunoassay to measure the antibody response against the repeat region of the circumsporozoite protein of Plasmodium falciparum Malaria J (2016) 15 (1): 543 doi: 10.1186/s12936-016-1596-8.
Raghavan, et al. Antibodies to repeat-containing antigens in Plasmodium falciparum are exposure-dependent and short-lived in children in natural malaria infections eLife (2023) 12: e81401 doi: 10.7554/eLife.81401.
Regules, et al. Fractional third and fourth dose of rts, S/as01 malaria candidate vaccine: A phase 2a controlled human malaria parasite infection and immunogenicity study J Infect Dis (2016) 214 (5): 762-71 doi: 10.1093/infdis/jiw237.
RTS SCTP. Efficacy and safety of rts, S/as01 malaria vaccine with or without a booster dose in infants and children in Africa: final results of a phase 3, individually randomised, controlled trial Lancet (2015) 386 (9988): 31-45 doi: 10.1016/s0140-6736 (15) 60721-8.
Ryan, et al. Comparison of major, minor and junctional circumsporozoite protein epitopes for malaria vaccine design. NPJ Vaccines. 2025 Oct. 3; 10 (1): 215. doi: 10.1038/s41541-025-01264-0.
Scally, et al. Rare pfcsp C-terminal antibodies induced by live sporozoite vaccination are ineffective against malaria infection J Exp Med (2018) 215 (1): 63-75 doi: 10.1084/jem.20170869.
Schwenk, et al. Igg2 Antibodies against a Clinical Grade Plasmodium Falciparum CSP Vaccine Antigen Associate with Protection against Transgenic Sporozoite Challenge in Mice PloS One (2014) 9 (10): e111020 doi: 10.1371/journal.pone.0111020.
Seth, et al. Development of a self-assembling protein nanoparticle vaccine targeting Plasmodium falciparum Circumsporozoite Protein delivered in three Army Liposome Formulation adjuvants Vaccine, 35 (41), 5448-5454 doi: 10.1016/j.vaccine.2017.02.040.
Suscovich, et al. Mapping functional humoral correlates of protection against malaria challenge following rts, S/as01 vaccination Sci Transl Med (2020) 12 (553) doi: 10.1126/scitranslmed.abb4757.
Thai, et al. A high-affinity antibody against the csp N-terminal domain lacks Plasmodium falciparum inhibitory activity J Exp Med (2020) 217 (11): e20200061 doi: 10.1084/jem.20200061.
Triller, et al. Natural parasite exposure induces protective human anti-malarial antibodies Immunity (2017) 47 (6): 1197-209.e10 doi: 10.1016/j.immuni.2017.11.007.
Ubillos, et al. Baseline exposure, antibody subclass, and hepatitis B response differentially affect malaria protective immunity following rts, S/as01e vaccination in African children BMC Med (2018) 16 (1): 197 doi: 10.1186/s12916-018-1186-4.
Wang, et al. A potent anti-malarial human monoclonal antibody targets circumsporozoite protein minor repeats and neutralizes sporozoites in the liver Immunity (2020) 53 (4): 733-44.e8 doi: 10.1016/j.immuni.2020.08.014.
Wang, et al. Protective effects of combining monoclonal antibodies and vaccines against the Plasmodium falciparum circumsporozoite protein PloS Pathog (2021) 17 (12): e1010133 doi: 10.1371/journal.ppat.1010133.
White, et al. The relationship between rts,S vaccine-induced antibodies, cd4 (+) T cell responses and protection against Plasmodium falciparum infection PloS One (2013) 8 (4): e61395 doi: 10.1371/journal.pone.0061395.
WHO. World malaria report, 2022 Geneva/Switzerland: WHO (2022).
Wu, et al. Low-dose subcutaneous or intravenous monoclonal antibody to prevent malaria New Engl J Med (2022) 387 (5): 397-407 doi: 10.1056/NEJMoa2203067.
Young, et al. Comprehensive data integration approach to assess immune responses and correlates of rts,S/as01-mediated protection from malaria infection in controlled human malaria infection trials Front Big Data (2021) 4:672460 doi: 10.3389/fdata.2021.672460.

All scientific and technical terms used in this application have meanings commonly used in the art unless otherwise specified.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates (NHPs), horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

As used herein, “antibody” refers to naturally occurring and synthetic immunoglobulin molecules and immunologically active portions thereof (i.e., molecules that contain an antigen binding site that specifically bind the molecule to which antibody is directed against). As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include: single chain Fvs (scFvs), Fab fragments, Fab′ fragments, F(ab′)₂, disulfide linked Fvs (sdFvs), Fvs, fragments comprising or alternatively consisting of, either a VL or a VH domain, and the like.

As used herein, a compound (e.g., receptor or antibody) “specifically binds” a given target (e.g., ligand or epitope) if it reacts or associates more frequently, more rapidly, with greater duration, and/or with greater binding affinity with the given target than it does with a given alternative, and/or indiscriminate binding that gives rise to non-specific binding and/or background binding. As used herein, “non-specific binding” and “background binding” refer to an interaction that is not dependent on the presence of a specific structure (e.g., a given target epitope). An antibody that specifically binds a given target over a specified alternative is an antibody that binds the given target with greater affinity, greater avidity, more readily, and/or with greater duration than it does to the specified alternative. An antibody that specifically binds a given epitope of, e.g., a given pathogen, is an antibody that binds the given epitope with greater affinity, greater avidity, more readily, and/or with greater duration than it does to other epitopes of the given pathogen and/or different species or variants of the given pathogen.

As used herein, an “epitope” and “antigenic epitope” are used interchangeably to refer to the part of a molecule that is recognized and specifically bound by an antibody. Epitopes may be linear epitopes or three-dimensional epitopes. As used herein, the terms “linear epitope” and “sequential epitope” are used interchangeably to refer to a primary structure of an antigen, e.g., a linear sequence of consecutive amino acid residues, that is recognized by an antibody. As used herein, the terms “three-dimensional epitope” and “conformational epitope” are used interchangeably to refer a three-dimensional structure that is recognized by an antibody, e.g., a plurality of non-linear amino acid residues that together are presented in three-dimensional space as they are in the antigen when in its native tertiary structure. That is, a conformational epitope may be synthetically created whereby the amino acid residues that are key to antibodies recognizing the native conformational epitope of the antigen are similarly presented in three-dimensional space whereby antibodies that specifically bind the synthetically created conformational epitope will also specifically bind the native conformational epitope presented on the antigen.

As used herein, “binding affinity” refers to the propensity of a compound to associate with (or alternatively dissociate from) a given target and may be expressed in terms of its dissociation constant, Kd. In some embodiments, the antibodies have a Kd of 10⁻⁵ or less, 10⁻⁶ or less, preferably 10⁻⁷ or less, more preferably 10⁻⁸ or less, even more preferably 10⁻⁹ or less, and most preferably 10⁻¹⁰ or less, to their given target. As used herein, “high affinity” and “high binding affinity” are used interchangeably herein to refer to an antibody that binds its given target with an affinity of 10⁻⁶ Kd or less. In some embodiments, the binding affinity of one or more benchmark antibodies to their cognate epitope employed in an ACE assay is 10⁻⁶ Kd or less. In some embodiments, the binding affinity of one or more benchmark antibodies to their cognate epitope employed in an ACE assay is 10⁻⁷ Kd or less. In some embodiments, the binding affinity of one or more benchmark antibodies to their cognate epitope employed in an ACE assay is 10⁻⁸ Kd or less. In some embodiments, the binding affinity of one or more benchmark antibodies to their cognate epitope employed in an ACE assay is 10⁻⁹ Kd or less. In some embodiments, the binding affinity of one or more benchmark antibodies to their cognate epitope employed in an ACE assay is 10⁻¹⁰ Kd or less.

Binding affinity can be determined using methods in the art, such as equilibrium dialysis, equilibrium binding, gel filtration, immunoassays, surface plasmon resonance, and spectroscopy using experimental conditions that exemplify the conditions under which the compound and the given target may come into contact and/or interact. Dissociation constants may be used determine the binding affinity of a compound for a given target relative to a specified alternative. Alternatively, methods in the art, e.g., immunoassays, in vivo or in vitro assays for functional activity, etc., may be used to determine the binding affinity of the compound for the given target relative to the specified alternative. Thus, in some embodiments, the binding affinity of the antibody for the given target is at least 1-fold or more, preferably at least 5-fold or more, more preferably at least 10-fold or more, and most preferably at least 100-fold or more than its binding affinity for the specified alternative.

As used herein, the term “sample” is used in its broadest sense and includes specimens and cultures obtained from any source, as well as biological samples and environmental samples. Biological samples may be obtained from animals (including humans) and encompass fluids, solids, tissues, and gases. Biological samples include blood products, such as plasma, serum, and the like. A biological sample can be obtained from a subject using methods in the art. A sample to be analyzed using one or more methods described herein can be either an initial unprocessed sample taken from a subject or a subsequently processed, e.g., partially purified, diluted, concentrated, fluidized, pretreated with a reagent (e.g., protease inhibitor, anti-coagulant, etc.), and the like. In some embodiments, the sample is a blood sample. In some embodiments, the blood sample is a whole blood sample, a serum sample, or a plasma sample. In some embodiments, the sample may be processed, e.g., condensed, diluted, partially purified, and the like. In some embodiments, the sample is pretreated with a reagent, e.g., a protease inhibitor. In some embodiments, two or more samples are collected at different time intervals to assess any difference in the amount of the analyte of interest, the progression of a disease or disorder, or the efficacy of a treatment, e.g., the strength of one's immunity against a given pathogen or antigen thereof. The test sample is then contacted with a capture reagent and, if the analyte is present, a conjugate between the analyte and the capture reagent is formed and is detected and/or measured with a detection reagent. In some embodiments, the sample to be tested is concentrated (or diluted) and then the level of a given antigen of interest (e.g., the CSP molecule) or one or more epitopes thereof is measured in the concentrated (or diluted) sample and the level in the concentrated (or diluted) sample is then mathematically extrapolated from the degree of concentration or dilution.

As used herein, a “capture reagent” refers to a molecule which specifically binds an analyte of interest. The capture reagent may be immobilized on a assay substrate. For example, if the analyte of interest is an antibody, the capture reagent may be an antigen or an epitope thereof to which the antibody specifically binds.

As used herein, an “assay substrate” refers to any substrate that may be used to immobilize a capture reagent thereon and then detect an analyte when bound thereto. Examples of assay substrates include membranes, beads, slides, and multi-well plates. As used herein, an assay well, in the singular, is a spot on an assay substrate to which a single aliquot of a test sample is assayed. A single assay well may comprise several different capture reagents, which each may be immobilized at its own given location in the single assay well, whereby all the different capture reagents in the same single assay well are contacted with the test sample when added (e.g., in the form of a test-reporter mixture) to the single assay well.

As used herein, a “detection reagent” refers to a substance that has a detectable label attached thereto and specifically binds an analyte of interest or a conjugate of the analyte of interest, e.g., an antibody-analyte conjugate.

As used herein, a “detectable label” is a compound or composition that produces or can be induced to produce a signal that is detectable by, e.g., visual, spectroscopic, photochemical, biochemical, immunochemical, or chemical means. While luminescent labels were used to detect the presence and amount of benchmark antibodies in the ACE assays herein, any detectable label in the art that allows the detection and quantification of different antibodies and/or epitope-antibody conjugates may be employed. The use of the term “labeled” as a modifier of a given substance, e.g., a labeled antibody, means that the substance has a detectable label attached thereto. A detectable label can be attached directly or indirectly by way of a linker (e.g., an amino acid linker or a chemical moiety). Examples of detectable labels include radioactive and non-radioactive isotopes (e.g., 125I, 18F, 13C, etc.), enzymes (e.g., β-galactosidase, peroxidase, etc.) and fragments thereof, enzyme substrates, enzyme inhibitors, coenzymes, catalysts, fluorophores (e.g., rhodamine, fluorescein isothiocyanate, etc.), dyes, chemiluminescers and luminescers (e.g., dioxetanes, luciferin, etc.), and sensitizers. A substance, e.g., antibody, having a detectable label means that a detectable label that is not linked, conjugated, or covalently attached to the substance, in its naturally-occurring form, has been linked, conjugated, or covalently attached to the substance by the hand of man. As used herein, the phrase “by the hand of man” means that a person or an object under the direction of a person (e.g., a robot or a machine operated or programmed by a person), not nature itself, has performed the specified act. Thus, the steps set forth in the claims are performed by the hand of man, e.g., a person or an object under the direction of the person.

A “vaccine” is a composition that induces a protective immune response in a subject when administered thereto. The protective immune response may be complete or partial, e.g., a reduction in symptoms caused by a given pathogen and/or a reduction in the degree of infection (i.e., titer of a given infectious pathogen) as compared with an unvaccinated subject.

As used herein, an “adjuvant” refers to any substance which, when administered in conjunction with (e.g., before, during, or after) a pharmaceutically active agent, such as one or more antigenic epitopes as disclosed herein, aids the pharmaceutically active agent in its mechanism of action, e.g., eliciting a protective immune response.

The test samples, reference samples, reporter mixtures and reference-reporter mixtures described herein may include buffers, preservatives, test agents (e.g., for determining whether they interfere with or enhance the binding of the antibodies to their target epitopes), etc.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient. For example, in the context of a composition “consisting essentially of” a given antibody that specifically binds a given epitope means that the composition may include additional antibodies and/or ingredients so long as they do not adversely affect, the ability of the given antibody to recognize and specifically bind its given epitope.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

As used herein, the terms “protein”, “polypeptide” and “peptide” are used interchangeably to refer to two or more amino acids linked together. Groups or strings of amino acid abbreviations are used to represent peptides. Except when specifically indicated, peptides are indicated with the N-terminus on the left and the sequence is written from the N-terminus to the C-terminus. Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A universal species-independent method for characterizing unknown antibodies that may be present in a test sample, which comprises
(a) selecting a plurality of epitopes of a given antigen of interest, wherein one or more epitopes in the plurality of epitopes are known to induce a protective antibody response against the given antigen in a subject when the subject is vaccinated with the one or more epitopes;
(b) obtaining a reporter mixture comprising a plurality of benchmark antibodies, each capable of binding one or more epitopes in the plurality of epitopes when contacted therewith;
(c) obtaining an assay substrate comprising the plurality of epitopes immobilized on a first single assay well, wherein the epitopes immobilized on the first single assay well are capable of being distinguished from each other;
(d) mixing the test sample with an amount of the reporter mixture to give a test mixture;
(e) contacting the plurality of epitopes immobilized on the first single assay well with the test mixture and then detecting the formation of the one or more epitope-antibody conjugates; and
(f) characterizing the unknown antibodies in the test sample as being protective when the unknown antibodies equally compete with or outcompete the plurality of benchmark antibodies in the amount of the reporter mixture in forming epitope-antibody conjugates with the one or more epitopes known to induce a protective antibody response that are immobilized on the first single assay well.

2. The method according to claim 1, wherein the concentration of each benchmark antibody in the plurality of benchmark antibodies in the reporter mixture does not exceed its linear detection range, wherein the linear detection range of a given antibody is a concentration range in which equal competition between the given antibody and itself for binding its cognate ligand is detectable.

3. The method according to claim 2, wherein the linear detection range of each benchmark antibody is determined by conducting a plurality of competition assay experiments between benchmark antibody test mixtures and at least one benchmark antibody reporter mixture, wherein the benchmark antibody test mixtures have differing concentrations of the plurality of benchmark antibodies, and wherein the benchmark antibodies in the benchmark antibody reporter mixture are distinguishable from the benchmark antibodies in the benchmark antibody test mixtures.

4. The method according to claim 1, wherein step (f) comprises comparing the formed epitope-antibody conjugates with a reference.

5. The method according to claim 4, wherein the reference is the amounts of each benchmark antibody in the plurality of benchmark antibodies that equally compete with themselves for binding each epitope immobilized on a second single assay well, said second single assay well is a duplicate of the first single assay well.

6. The method according to claim 4, wherein the reference is a profile of epitope-antibody conjugates formed between antibodies of a reference-reporter mixture, said reference-reporter mixture comprising (1) a reference sample having the plurality of benchmark antibodies the same amount as in the reporter mixture that was mixed with the test sample, and (2) the same amount of the reporter mixture that was mixed with the test sample, wherein, when bound to one or more epitopes immobilized on second single assay well, said second single assay well is a duplicate of the first single assay well, the benchmark antibodies from the reference sample are distinguishable from the benchmark antibodies from the reporter mixture.

7. The method according to claim 6, which further comprises
(i) contacting the reference-reporter mixture with the plurality of epitopes immobilized on the second single assay well; and
(ii) comparing the amounts of the benchmark antibodies from the reporter mixture that are bound to each epitope in the plurality of epitopes immobilized in the first single assay well to the amounts of either the benchmark antibodies of the reference sample or the benchmark antibodies in the reporter mixture that are bound to the plurality of epitopes immobilized on the second single assay well.

8. The method according to claim 1, which further comprises obtaining a standard curve for each epitope in the plurality of epitopes, wherein the standard curve of a given epitope represents the amounts of the plurality of the benchmark antibodies in the reporter mixture that outcompete themselves over several different concentrations and/or dilutions of the concentration of the plurality of the benchmark antibodies present in the reporter mixture and thereby bind the given epitope that is immobilized in single assay wells that are duplicates of the first single assay well.

9. The method according to claim 1, which further comprises obtaining a dose response curve of the test sample that represents different dilutions and/or concentrations of the test sample titrated against the reporter mixture.

10. The method according to claim 1, which further comprises obtaining a dose response curve of several different concentrations and/or dilutions of a reference sample titrated against the reporter mixture, wherein said reference sample comprises the same benchmark antibodies as in the reporter mixture and in the same concentrations.

11. The method according to claim 1, wherein each benchmark antibody in the plurality of benchmark antibodies in the reporter mixture have a detectable label.

12. A method of obtaining an antibody profile of a subject, which comprises performing the method according to claim 1 on a sample obtained from the subject that is used as the test sample.

13. The method according to claim 12, which further comprises administering to the subject a composition comprising the given antigen of interest or an antigenic epitope thereof before and/or after obtaining the sample from the subject.

14. The method according to claim 12, which comprises challenging the subject with a pathogen that expresses the given antigen of interest before and/or after obtaining the sample from the subject.

15. The method according to claim 13, which comprises challenging the subject with a pathogen that expresses the given antigen of interest before and/or the subject is administered the composition.

16. A kit comprising
(a) (1) a plurality of antigenic epitopes of a given antigen, which each antigenic epitope is immobilized in an assigned location in each assay well of an assay substrate whereby each single assay well contains the plurality of antigenic epitopes immobilized at their own assigned location; or (2) the plurality of antigenic epitopes and linkers along with reagents for immobilizing each antigenic epitope in the plurality at an assigned location in each assay well of the assay substrate;
(b) a reporter mixture comprising a plurality of benchmark antibodies; and
(c) a reference.

17. The kit according to claim 16, wherein the reference is a reference sample.


SEQUENCE LISTING (7 sequences)

SEQ ID NO: 1 - protein, 74 aa, synthetic construct
GSSSNTRVLNELNYDNAGTNLYNELEMNYYGKQENWYSLKKNSRSLGENDDGNNEDNEKLRKPKHKKLKQPADG

SEQ ID NO: 2 - protein, 18 aa, synthetic construct
KQPADGNPDPNANPNVDP

SEQ ID NO: 3 - protein, 24 aa, synthetic construct
NVDPNANPNVDPNANPNVDPNANP

SEQ ID NO: 4 - protein, 24 aa, synthetic construct
NPNANPNANPNANPNANPNANPNA

SEQ ID NO: 5 - protein, 93 aa, synthetic construct
HNMPNDPNRNVDENANANSAVKNNNNEEPSDKHIKEYLNKIQNSLSTEWSPCSVTCGNGIQVRIKPGSANKPKDELDYANDIEKKICKMEKCS

SEQ ID NO: 6 - protein, 93 aa, synthetic construct
HNMPNDPNRNVDENANANNAVKNNNNEEPSDKHIKEYLNKIQNSISTEWSPCSVTCGNGIQVRIKPGSADKPKDQLDYINDIEKKICKMEKCS

SEQ ID NO: 7 - protein, 93 aa, synthetic construct
HNMPNDPNRNVDENANANNAVKNNNNEEPSDKHIEQYLKTIKNSLSTEWSPCSVTCGNGIQVRIKPGSAGKSKNELDYENDIEKKICKMEKCS